SEQUENCE LISTING (2 sequences)

SEQ ID NO: 1 - protein, 213 aa, synthetic construct
  REGION 1..213: note = Synthetic
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
  REGION 1..449: note = Synthetic
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0409633 A1
(43) Pub. Date: Dec. 12, 2024

(54) Methods and Compositions for Preventing Type 1 Diabetes

(71) Applicants: Provention Bio, Inc., Red Bank, NJ (US); Benaroya Research Institute at Virginia Mason, Seattle, WA (US); Yale University, New Haven, CT (US)

(72) Inventors: Francisco LEON, Bethesda, MD (US); Kevan C. HEROLD, Norwalk, CT (US); Sarah Alice LONG, Seattle, WA (US); Peter S. Linsley, Seattle, WA (US)

(73) Assignees: Provention Bio, Inc., Red Bank, NJ (US); Benaroya Research Institute at Virginia Mason, Seattle, WA (US); Yale University, New Haven, CT (US)

(21) Appl. No.: 18/666,000

(22) Filed: May 16, 2024

Related U.S. Application Data
(62) Division of application No. 17/345,495, filed on Jun. 11, 2021, now Pat. No. 12,006,366.
(60) Provisional application No. 63/037,968, filed on Jun. 11, 2020.
(60) Provisional application No. 63/192,242, filed on May 24, 2021.

(30) Foreign Application Priority Data
Jan. 26, 2021 (TW) 110102871

(51) Int. Cl. C07K 16/28; A61K 39/00; A61P 3/10; G01N 33/68

(52) U.S. Cl. CPC C07K 16/2809; A61P 3/10; G01N 33/6854; A61K 2039/505; A61K 2039/545; G01N 2800/52

(57) ABSTRACT

Provided herein, in one aspect, is a method of preventing or delaying the onset of clinical type 1 diabetes (T1D), comprising: providing a non-diabetic subject who is at risk for T1D; administering a prophylactically effective amount of an anti-CD3 antibody to the non-diabetic subject; and determining, prior to or after the administering step, that the non-diabetic subject has more than about 5% to more than about 10% TIGIT+KLRG1+CD8+ T-cells in all CD3+ T-cells, which is indicative of successful prevention or delay of the onset of clinical T1D.

### CROSS REFERENCE TO RELATED APPLICATIONS

This application is a divisional of U.S. application Ser. No. 17/345,495, filed Jun. 11, 2021, which claims priority to and the benefit of U.S. Provisional Patent Application No. 63/037,968, filed Jun. 11, 2020, Taiwanese Patent Application No. 110102871, filed Jan. 26, 2021, and U.S. Provisional Patent Application No. 63/192,242, filed May 24, 2021. All of the aforementioned priority applications are incorporated herein by reference in their entirety.

### SEQUENCE LISTING

The XML file submitted herewith via Patent Center, entitled “122548.D1053.xml,” created on May 2, 2024, and having a size of 3,959 bytes, is incorporated herein by reference in its entirety.

### FIELD

The present disclosure relates in general to compositions and methods of preventing or delaying the onset of clinical type 1 diabetes (T1D) in subjects at risk, and more particularly the use of anti-CD3 antibodies.

### BACKGROUND

Type 1 diabetes (T1D) is caused by the autoimmune destruction of insulin producing beta cells in the islets of Langerhans leading to dependence on exogeneous insulin injections for survival. Approximately 1.6 million Americans have Type 1 diabetes, and after asthma, it remains one of the most common diseases of childhood. Despite improvements in care, most affected individuals with T1D are not able to consistently achieve desired glycemic targets. For individuals with type 1 diabetes, there are persisting concerns for increased risk of both morbidity and mortality. Two recent studies noted loss of 17.7 life-years for children diagnosed before age 10, and 11 and 13 life-years lost for adult-diagnosed Scottish men and women respectively.

In genetically susceptible individuals, T1D progresses through asymptomatic stages prior to overt hyperglycemia, characterized first by the appearance of autoantibodies (Stage 1) and then dysglycemia (Stage 2). In Stage 2, metabolic responses to a glucose load are impaired but other metabolic indices, for example glycosylated hemoglobin, are normal and insulin treatment is not needed. These immunologic and metabolic features identify individuals who are at high-risk for development of clinical disease with overt hyperglycemia and requirement for insulin treatment (Stage 3). Several immune interventions have been shown to delay decline in beta cell function when studied in recent-onset clinical T1D. One promising therapy is the FcR non-binding anti-CD3 monoclonal antibody teplizumab, as several studies have shown that short-term treatment reduces loss of β cell function durably, with an observable effect seen as long as 7 years after diagnosis and treatment. The drug modifies the function of CD8+ T lymphocytes, which are thought to be important effector cells that cause beta cell killing.

To date, no intervention initiated before the clinical diagnosis (i.e., at Stage 1 or 2) has altered progression to clinical, Stage 3 T1D. Thus, a need exists for a treatment that would prevent or delay the onset of clinical T1D in high-risk individuals.

### SUMMARY

A method of preventing or delaying the onset of clinical type 1 diabetes (T1D), comprising:
providing a non-diabetic subject who is at risk for T1D;
administering a prophylactically effective amount of an anti-CD3 antibody to the non-diabetic subject; and
determining, prior to or after the administering step, that the non-diabetic subject has more than about 5% to more than about 10% TIGIT+KLRG1+CD8+ T-cells in all CD3+ T-cells, which is indicative of successful prevention or delay of the onset of clinical T1D.

In some embodiments, the non-diabetic subject is a relative of a patient with T1D.

In some embodiments, the method further includes determining that the non-diabetic subject (1) is substantially free of antibodies against zinc transporter 8 (ZnT8), (2) is HLA-DR4+, and/or (3) is not HLA-DR3+.

In some embodiments, the non-diabetic subject has 2 or more diabetes-related autoantibodies selected from islet cell antibodies (ICA), insulin autoantibodies (IAA), and antibodies to glutamic acid decarboxylase (GAD), tyrosine phosphatase (IA-2/ICA512) or ZnT8.

In some embodiments, the non-diabetic subject has abnormal glucose tolerance on oral glucose tolerance test (OGTT). In some embodiments, the abnormal glucose tolerance on OGTT is a fasting glucose level of 110-125 mg/dL, or 2 hour plasma of ≥140 and <200 mg/dL, or an intervening glucose value at 30, 60, or 90 minutes on OGTT >200 mg/dL.

In some embodiments, the non-diabetic subject does not have antibodies against ZnT8.

In some embodiments, the non-diabetic subject is HLA-DR4+ and is not HLA-DR3+.

In some embodiments, the anti-CD3 antibody is selected from teplizumab, otelixizumab or foralumab. In some embodiments, the prophylactically effective amount of the antibody comprises a 10 to 14 day course of subcutaneous (SC) injection or intravenous (IV) infusion or oral administration of the anti-CD3 antibody at 10-1000 micrograms/meter squared (μg/m²), preferably a 14-day course IV infusion at 51 μg/m², 103 μg/m², 207 μg/m², and 413 μg/m², on days 0-3, respectively, and one dose of 826 μg/m² on each of days 4-13.

In some embodiments, the prophylactically effective amount delays median time to clinical diagnosis of T1D by at least 50%, at least 80%, or at least 90%, or at least 12 months, at least 18 months, at least 24 months, at least 36 months, at least 48 months, or at least 60 months.

In some embodiments, the determining of TIGIT+KLRG1+CD8+ T-cells is by flow cytometry.

In some embodiments, the method further includes determining a decrease in a percentage of CD8+ T-cells expressing proliferation markers Ki67 and/or CD57.

A method of prognosing responsiveness of an anti-CD3 antibody in preventing or delaying the onset of type 1 diabetes (T1D), comprising:
providing a non-diabetic subject who is at risk for T1D;
administering a prophylactically effective amount of an anti-CD3 antibody to the non-diabetic subject; and
determining C-peptide area under the curve (AUC): glucose AUC ratio, wherein an increase in said ratio indicates responsiveness to the anti-CD3 antibody and/or non-progression to clinical T1D.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a consort diagram of at-risk individuals enrolled into the teplizumab prevention study.

FIG. 1B shows that teplizumab treatment is associated with a sustained effect on Type 1 Diabetes progression over 923 days of follow-up. Updated Kaplan-Meier Curve based on 923 days of follow up (range 74-3,119 days). Hazard ratio for development of type 1 diabetes in teplizumab-treated participants vs. placebo was 0.457; p=0.01. The median time to diabetes was 24.4 (or 27.1 months according to Sims et al., Sci Transl Med. (2021) 13:eabc8980, incorporated herein by reference) and 59.6 months in the placebo and teplizumab treatment groups respectively. Updated At the conclusion of this period, 7 (22%) and 22 (50%) respectively were not diagnosed with T1D.

FIGS. 2A and 2B show results of OGTT tests over the first 36 months in (FIG. 2A) teplizumab and (FIG. 2B) placebo treated participants: each line represents a participant. The symbols indicate the time of OGTT testing. The results are indicated as: black dot=diabetic level/diagnosis, red dot=dysglycemia, blue dot=normal.

FIGS. 3A and 3B show that improved glycemia in teplizumab treated participants is associated with maintenance of dysglycemic status. FIG. 3A shows OGTT classifications for participants in each group over 36 months of follow-up. The data are shown to 36 months because of loss of placebo treated participants because of the clinical diagnosis of T1D (for individual participants see FIGS. 2A-2B). FIG. 3B shows a boxplot displaying median and interquartile ranges for on-study OGTT glucose AUC mean for participants from placebo and teplizumab treated groups. ANCOVA model incorporating baseline value, age, and treatment group showed that treatment group had a significant effect to decrease average on-study glucose AUC (ANCOVA teplizumab effect: 92.8%, p=0.02).

FIG. 4 shows the average on-study Hemoglobin A1c levels in the treatment groups in a boxplot displaying median and interquartile ranges for average on-study Hemoglobin A1c AUC for participants from placebo and teplizumab treated groups. ANCOVA model incorporating baseline value, age, and treatment group showed no significant impact of treatment group (p=0.14).

FIG. 5 shows that teplizumab treatment was associated with increased average on-study C-peptide AUC. FIG. 5 shows a boxplot displaying median and interquartile ranges for average on-study OGTT C-peptide AUC mean for participants from placebo and teplizumab treated groups. An ANCOVA model including baseline C-peptide AUC and age showed that treatment was associated with higher average on-study C-peptide AUC (p=0.009).

FIG. 6 shows the relationship of average on-study C-peptide AUC with age and average on-study glucose AUC. FIG. 6 shows a scatterplot of age vs. on-study C-peptide AUC (r=0.44, p=0.0001).

FIG. 7 shows C-peptide over time in the two treatment arms over the first year. The log-transformed mean C-peptide AUC is shown. Arrows indicate individual drop out from OGTT monitoring due to diabetes development after each timepoint. Median value for “pre-baseline” timepoint was 24. Months prior to randomization and median value for “baseline” timepoint was 0.85 months prior to randomization. *P<0.05 for comparisons of 6-month on-treatment C-peptide AUC values to baseline in the teplizumab group and 6-month C-peptide AUC values in the teplizumab group to 6-month C-peptide AUC values in the placebo group.

FIGS. 8A-8G show insulin secretion following treatment with teplizumab or placebo. Estimated slopes for the insulin secreted (pmol) secreted during the total (FIG. 8A), first hour (FIG. 8B), and second hour (FIG. 8C) of the OGTT at the visits before enrollment and over the first 6 months following study drug treatment. Median values (and 95% CIs in shaded colors) are shown. Refer to Table 5 for statistical analyses. FIGS. 8D and 8E show representative insulin secretion rates during serial OGTTs for 2 teplizumab participants who were not diagnosed with T1D (age 11 and 12 years) and (FIGS. 8F and 8G) two placebo treated individuals (both age 13 years) who were diagnosed with T1D. The color lines indicate the time of the visits in relationship to study drug administration.

FIGS. 9A-9E show that teplizumab preserves C-peptide over the course of the study until the period surrounding diagnosis. For all panels, data from teplizumab-treated participants are shown in blue, and placebo-treated participants are shown in maroon. FIG. 9A and FIG. 9B show regression lines for C-peptide AUC values over the study period of OGTT monitoring from baseline study visit until diagnosis (teplizumab n=44, placebo n=32). FIGS. 9C and 9D show regression lines for C-peptide AUC values over 6-month period before diabetes diagnosis (placebo n=23, teplizumab n=22). FIG. 9E shows slopes of C-peptide AUC for 6-month period before diagnosis in those that developed T1D, and the last 6 months of study in individuals remaining T1D-free.

FIGS. 10A and 10B show that C-peptide values are similar between treatment groups at the time of diagnosis. FIG. 10A shows C-peptide AUC/Glucose AUC at the time of clinical diagnosis of T1D. FIG. 10B shows C-peptide AUC at the time of clinical diagnosis of T1D. Values were obtained from the first of two consecutive diagnostic OGTTs consistent with a classification of T1D.

FIGS. 11A-11C show that functional changes in T-cells are associated with improvements in metabolic function. FIG. 11A shows the changes in TIGIT+KLRG1+CD45RO+CD8+ T-cells between baseline and 3 months and the change in the C-peptide AUC between the baseline and 6 months are shown. There was a significant correlation between the changes in this cell subset and C peptide in the teplizumab (Pearson r=0.44, p=0.014, n=31) but not the placebo treated (r=0.28, p=0.25, n=18) participants. FIGS. 11B and 11C show the frequency of double positive (DP, i.e. TIGIT+KLRG1+) CD8+ memory cells that produce IFNγ or TNFα are shown for the placebo treated (red dot, n=16) and drug treated (blue dot, n=24) participants at baseline and month 3. The frequency of the IFNγ and TNFα producing cells were reduced in the teplizumab treated participants (paired T-test, ***p<0.0001).

FIG. 12 shows teplizumab treated subjects with the best outcome have more TIGIT+KLRG1+ CD8 T-cells.

FIGS. 13 and 14 show total TIGIT+KLRG1+ CD8 T-cells resemble exhausted cells.

FIGS. 15 and 16 show total TIGIT+KLRG1+ CD8 T-cells are heterogeneous.

FIG. 17 shows TIGIT+KLRG1+ T-cells are spread across much of the memory CD8 landscape in the teplizumab TrialNet Stage 2 trial.

FIGS. 18-21 show TIGIT+KLRG1+ subsets differ in response and outcome.

FIG. 22 shows OGTT patterns of 30 to 120 minute glucose and C-peptide.

FIG. 23 shows 2D plots of mean glucose and C-peptide values identify distinct longitudinal OGTT patterns among “Progressors” and “NonProgressors” within DPT-1.

FIG. 24 shows 2D plots of mean glucose and C-peptide values for 30-120 minute OGTT timepoints show distinct patters at 3 month visit.

FIG. 25 shows 3-month change in ratio of C-peptide AUC/Glucose AUC shows most significant differences compared to other metabolic measures.

FIG. 26 shows Predicted Mean Teplizumab Serum Concentration Versus Time Profile Following 14-Day Regimen Across Different Body Weights.

### DETAILED DESCRIPTION

Provided herein, in some embodiments, is a method of preventing or delaying the onset of clinical type 1 diabetes (T1D), comprising: providing a non-diabetic subject who is at risk for T1D; administering a prophylactically effective amount of an anti-CD3 antibody to the non-diabetic subject; and determining, prior to or after the administering step, that the non-diabetic subject has more than about 5% to more than about 10% TIGIT+KLRG1+CD8+ T-cells in all CD3+ T-cells, which is indicative of successful prevention or delay of the onset of clinical T1D.

In some embodiments, a method of prognosing responsiveness of an anti-CD3 antibody, e.g., teplizumab, in preventing or delaying the onset of T1D is provided. The method can include: providing a non-diabetic subject who is at risk for T1D; administering a prophylactically effective amount of the anti-CD3 antibody, e.g., teplizumab, to the non-diabetic subject; and determining C-peptide area under the curve (AUC): glucose AUC ratio, wherein an increase in said ratio indicates responsiveness to the anti-CD3 antibody.

### Definitions

Certain terms are defined herein below. Additional definitions are provided throughout the application.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

As used herein, the term “prophylactic agent” refers to CD3 binding molecules such as teplizumab which can be used in the prevention, treatment, management or amelioration of one or more symptoms of T1D.

As used herein, the term “onset” of disease with reference to Type-1 diabetes refers to a patient meeting the criteria established for diagnosis of Type-1 diabetes by the American Diabetes Association (see Mayfield et al., Am Fam Physician (2006) 58:1355-1362).

As used herein, the terms “prevent,” “preventing” and “prevention” refer to the prevention of the onset of one or more symptoms of T1D in a subject resulting from the administration of a prophylactic or therapeutic agent.

As used herein, a “protocol” includes dosing schedules and dosing regimens. The protocols herein are methods of use and include prophylactic and therapeutic protocols. A “dosing regimen” or “course of treatment” may include administration of several doses of a therapeutic or prophylactic agent over 1 to 20 days.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey or a human), and more preferably a human.

As used herein, the term “prophylactically effective amount” refers to that amount of teplizumab sufficient to result in the delay or prevention of the development, recurrence or onset of one or more symptoms of T1D. In some embodiments, a prophylactically effective amount preferably refers to the amount of teplizumab that delays a subject's onset of T1D by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%.

Various aspects of the disclosure are described in further detail below. Additional definitions are set out throughout the specification.

### Anti-CD3 Antibodies and Pharmaceutical Compositions

The terms “anti-CD3 antibody” and “an antibody that binds to CD3” refer to an antibody or antibody fragment that is capable of binding cluster of differentiation 3 (CD3) with sufficient affinity such that the antibody is useful as a prophylactic, diagnostic and/or therapeutic agent in targeting CD3. In some embodiments, the extent of binding of an anti-CD3 antibody to an unrelated, non-CD3 protein is less than about 10% of the binding of the antibody to CD3 as measured, e.g., by a radioimmunoassay (RIA). In some embodiments, an antibody that binds to CD3 has a dissociation constant (K_{d}) of <1 μM, <100 nM, <10 nM, <1 nM, <0.1 nM, <0.01 nM, or <0.001 nM (e.g., 10⁻8 M or less, e.g., from 10⁻⁸ M to 10⁻¹³ M, e.g., from 10⁻⁹ M to 10⁻¹³ M). In some embodiments, an anti-CD3 antibody binds to an epitope of CD3 that is conserved among CD3 from different species.

In some embodiments, the anti-CD3 antibody can be ChAglyCD3 (otelixizumab). Otelixizumab is a humanized Fc nonbinding anti-CD3, which was evaluated initially in phase 2 studies by the Belgian Diabetes Registry (BDR) and then developed by Tolerx, which then partnered with GSK to conduct the phase 3 DEFEND new onset T1D trials (NCT00678886, NCT01123083, NCT00763451). Otelixizumab is administered IV with infusions over 8 days. See, e.g., Wiczling et al., J Clin Pharmacol. (2010) 50(5):494-506; Keymeulen et al., N Engl J Med. (2005) 352:2598-608; Keymeulen et al., Diabetologia (2010) 53:614-23; Hagopian et al., Diabetes (2013) 62:3901-8; Aronson et al., Diabetes Care (2014) 37:2746-54; Ambery et al., Diabet Med. (2014) 31:399-402; Bolt et al., Eur J Immunol. 1YY3. 23:403-411; Vlasakakis et al., Br J Clin Pharmacol (2019) 85:704-14; Guglielmi et al., Expert Opinion on Biological Therapy 16(6):841-6; Keymeulen et al., N Engl J Med. (2005) 352:2598-608; Keymeulen et al., Blood (2010) 115(6); Sprangers et al., Immunotherapy (2011) 3(11):1303-16; Daifotis et al., Clinical Immunology (2013) 149:268-78; all incorporated herein by reference.

In some embodiments, the anti-CD3 antibody can be visilizumab (also called HuM291;Nuvion). Visilizumab is a humanized anti-CD3 monoclonal antibody characterized by a mutated IgG2 isotype, lack of binding to Fcγ receptors, and the ability to induce apoptosis selectively in activated T-cells. It was evaluated in patients in graft-versus-host disease (NCT00720629; NCT00032279) and in ulcerative colitis (NCT00267306) and Crohn's Disease (NCT00267709). See, e.g., Sandborn et al., Gut (2010) 59(11):1485-92, incorporated herein by reference.

In some embodiments, the anti-CD3 antibody can be foralumab, a fully human anti-CD3 monoclonal antibody being developed by Tiziana Life Sciences, PLC in NASH and T2D (NCT03291249). See, e.g., Ogura et al., Clin Immunol. (2017) 183:240-6; Ishikawa et al., Diabetes (2007) 56(8):2103-9; Wu et al., J Immunol. (2010) 185(6):3401- 7; all incorporated herein by reference.

In some embodiments, the anti-CD3 antibody can be teplizumab. Teplizumab, also known as hOKT3yl(Ala-Ala) (containing an alanine at positions 234 and 235) is an anti-CD3 antibody that had been engineered to alter the function of the T lymphocytes that mediate the destruction of the insulin-producing beta cells of the islets of the pancreas. Teplizumab binds to an epitope of the CD3ε chain expressed on mature T-cells and by doing so changes their function. Sequences and compositions of teplizumab are disclosed in U.S. Pat. Nos. 6,491,916; 8,663,634; and 9,056,906, each incorporated herein by reference in its entirety. The full sequences of light and heavy chains are set forth below. Bolded portions are the complementarity determining regions.

Teplizumab Light Chain (SEQ ID NO: 1):
DIQMTQSPSSLSASVGDRVTITCSASSSVSYMNWY
QQTPGKAPKRWIYDTSKLASGVPSRFSGSGSGTDY
TFTISSLQPEDIATYYCQQWSSNPFTFGQGTKLQI
TRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC
Teplizumab Heavy Chain (SEQ ID NO: 2):
QVQLVQSGGGVVQPGRSLRLSCKASGYTFTRYTMH
WVRQAPGKGLEWIGYINPSRGYTNYNQKVKDRFTI
SRDNSKNTAFLQMDSLRPEDTGVYFCARYYDDHYC
LDYVVGQGTPVTVSSASTKGPSVFPLAPSSKSTSG
GTAALGCLVKDYFPEPVTVSWNSGALTSGVHTFPA
VLQSSGLYSLSSVVTVPSSSLGTQTYICNVNHKPS
NTKVDKKVEPKSCDKTHTCPPCPAPEAAGGPSVFL
FPPKPKDTLMISRTPEVTCVVVDVSHEDPEVKFNW
YVDGVEVHNAKTKPREEQYNSTYRVVSVLTVLHQD
WLNGKEYKCKVSNKALPAPIEKTISKAKGQPREPQ
VYTLPPSRDELTKNQVSLTCLVKGFYPSDIAVEWE
SNGQPENNYKTTPPVLDSDGSFFLYSKLTVDKSRW
QQGNVFSCSVMHEALHNHYTQKSLSLSPGK

In some embodiments, provided herein, is a pharmaceutical composition. Such compositions comprise a prophylactically effective amount of an anti-CD3 antibody, and a pharmaceutically acceptable carrier. In some embodiments, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like (see, for example, Handbook of Pharmaceutical Excipients, Arthur H. Kibbe (ed., 2000, which is incorporated by reference herein in its entirety), Am. Pharmaceutical Association, Washington, D.C.

The composition, if desired, can also contain minor amounts of wetting or emulsifying agents, or pH buffering agents. These compositions can take the form of solutions, suspensions, emulsion, tablets, pills, capsules, powders, sustained release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In some embodiments, the pharmaceutical compositions are sterile and in suitable form for administration to a subject, preferably an animal subject, more preferably a mammalian subject, and most preferably a human subject.

In some embodiments, it may be desirable to administer the pharmaceutical compositions locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering the anti-CD3 antibody, care must be taken to use materials to which the anti-CD3 antibody does not absorb.

In some embodiments, the composition can be delivered in a vesicle, in particular a liposome (see Langer, Science (1990) 249:1527-33; Treat et al., in Liposomes in the Therapy of Infectious Disease and Cancer, Lopez-Berestein and Fidler (eds.), Liss, New York, pp. 353-65(1989); Lopez-Berestein, ibid., pp. 317-27; see generally ibid.).

In some embodiments, the composition can be delivered in a controlled release or sustained release system. In some embodiments, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., N Engl J Med. (1989) 321:574). In some embodiments, polymeric materials can be used to achieve controlled or sustained release of the antibodies of the invention or fragments thereof (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., Science (1985) 228:190; During et al., Ann Neurol. (1989) 25:351; Howard et al., J Neurosurg. (1989) 71:105); U.S. Pat. Nos. 5,679,377; 5,916,597;5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In some embodiments, the polymer used in a sustained release formulation is inert, free of leachable impurities, stable on storage, sterile, and biodegradable. In some embodiments, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-38 (1984)).

Controlled release systems are discussed in the review by Langer, Science (1990) 249:1527-33). Any technique known to one of skill in the art can be used to produce sustained release formulations comprising one or more antibodies of the invention or fragments thereof. See, e.g., U.S. Pat. No. 4,526,938; PCT Publication No. WO 91/05548; PCT Publication No. WO 96/20698; Ning et al., 1996, Radiotherapy & Oncology 39:179-89; Song et al., 1995, PDA Journal of Pharmaceutical Science & Technology 50:372-97; Cleek et al., 1997, Pro. Int'l. Symp. Control. Rel. Bioact. Mater. 24:853-4; and Lam et al., 1997, Proc. Int'l. Symp. Control Rel. Bioact. Mater. 24:759-60, each of which is incorporated herein by reference in its entirety.

A pharmaceutical composition can be formulated to be compatible with its intended route of administration. Examples of routes of administration include, but are not limited to, parenteral, e.g., intravenous, intradermal, subcutaneous, oral, intranasal (e.g., inhalation), transdermal (topical), transmucosal, and rectal administration. In some embodiments, the composition is formulated in accordance with routine procedures as a pharmaceutical composition adapted for intravenous, subcutaneous, intramuscular, oral, intranasal or topical administration to human beings. In some embodiments, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocaine to case pain at the site of the injection.

The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In some embodiments, the disclosure provides dosage forms that permit administration of the anti-CD3 antibody continuously over a period of hours or days (e.g., associated with a pump or other device for such delivery), for example, over a period of 1 hour, 2 hours, 3 hours, 4 hours, 6 hours, 8 hours, 10 hours, 12 hours, 16 hours, 20 hours, 24 hours, 30 hours, 36 hours, 4 days, 5 days, 7 days, 10 days or 14 days. In some embodiments, the invention provides dosage forms that permit administration of a continuously increasing dose, for example, increasing from 51 ug/m²/day to 826 ug/m²/day over a period of 24 hours, 30 hours, 36 hours, 4 days, 5 days, 7 days, 10 days or 14 days.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of the compositions disclosed herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the disclosure provides that the anti-CD3 antibodies, or pharmaceutical compositions thereof, can be packaged in a hermetically sealed container such as an ampoule or sachette indicating the quantity of the agent. In some embodiments, the anti-CD3 antibody, or pharmaceutical compositions thereof is supplied as a dry sterilized lyophilized powder or water free concentrate in a hermetically sealed container and can be reconstituted, e.g., with water or saline to the appropriate concentration for administration to a subject. Preferably, the anti-CD3 antibody, or pharmaceutical compositions thereof is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic agents, or pharmaceutical compositions herein should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In some embodiments, the pharmaceutical composition is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/ml, at least 25 mg/ml, at least 50 mg/ml, at least 75 mg/ml or at least 100 mg/ml. The liquid form should be stored at between 2° C. and 8° C. in its original container.

In some embodiments, the disclosure provides that the composition of the invention is packaged in a hermetically sealed container such as an ampoule or sachette indicating the quantity of the anti-CD3 antibody.

The compositions may, if desired, be presented in a pack or dispenser device that may contain one or more unit dosage forms containing the active ingredient. The pack may, for example, comprise metal or plastic foil, such as a blister pack.

The amount of the composition of the invention which will be effective in the prevention or amelioration of one or more symptoms associated with T1D can be determined by standard clinical techniques. The precise dose to be employed in the formulation will also depend on the route of administration and the seriousness of the condition, and should be decided according to the judgment of the practitioner and each patient's circumstances. Effective doses may be extrapolated from dose-response curves derived from in vitro or animal model test systems.

### Methods and Use

In some embodiments, the present disclosure encompasses administration of anti-human CD3 antibodies such as teplizumab to individuals predisposed to develop type 1 diabetes or with pre-clinical stages of type 1 diabetes, but who do not meet the diagnosis criteria as established by the American Diabetes Association or the Immunology of Diabetes Society to prevent or delay the onset of type 1 diabetes and/or to prevent or delay the need for administration of insulin to such patients. In some embodiments, high-risk factors for identification of predisposed subjects include having first or second degree relatives with diagnosed type-1 diabetes, an impaired fasting glucose level (e.g., at least one determination of a glucose level of 100-125 mg/dl after fasting (8 hours with no food)), an impaired glucose tolerance in response to a 75 g OGTT (e.g., at least one determination of a 2-hr glucose level of 140-199 mg/dl in response to a 75 g OGTT), an HLA type of DR3, DR4 or DR7 in a Caucasian, an HLA type of DR3 or DR4 in a person of African descent, an HLA type of DR3, DR4 or DR9 in a person of Japanese descent, exposure to viruses (e.g., coxsackie B virus, enteroviruses, adenoviruses, rubella, cytomegalovirus, Epstein-Barr virus), a positive diagnosis according to art accepted criteria of at least one other autoimmune disorder (e.g., thyroid disease, celiac disease), and/or the detection of autoantibodies, particularly ICAs and type 1 diabetes-associated autoantibodies, in the serum or other tissues. In some embodiments, the subject identified as predisposed to developing type 1 diabetes has at least one of the risk factors described herein and/or as known in the art. The present disclosure also encompasses identification of subjects predisposed to development of type 1 diabetes, wherein said subject presents a combination of two or more, three or more, four or more, or more than five of the risk factors disclosed herein or known in the art.

Serum autoantibodies associated with type 1 diabetes or with a predisposition for the development of type 1 diabetes are islet-cell autoantibodies (e.g., anti-ICA512 autoantibodies), glutamic acid decarbamylase autoantibodies (e.g., anti-GAD65 autoantibodies), IA2 antibodies, ZnT8 antibodies and/or anti-insulin autoantibodies. Accordingly, in a specific example in accordance with this embodiment, the invention encompasses the treatment of an individual with detectable autoantibodies associated with a predisposition to the development of type 1 diabetes or associated with early stage type 1 diabetes (e.g., anti-IA2, anti-ICA512, anti-GAD or anti-insulin autoantibodies), wherein said individual has not been diagnosed with type 1 diabetes and/or is a first or second degree relative of a type-1 diabetic. In some embodiments, the presence of the autoantibodies is detected by ELISA, electrochemoluminescence (ECL), radioassay (see, e.g., Yu et al., J Clin Endocrinol Metab. (1996) 81:4264-7), agglutination PCR (Tsai et al., ACS Central Science (2016) 2(3):139-47) or by any other method for immunospecific detection of antibodies described herein or as known to one of ordinary skill in the art.

β-cell function prior to, during, and after therapy may be assessed by methods described herein or by any method known to one of ordinary skill in the art. For example, the Diabetes Control and Complications Trial (DCCT) research group has established the monitoring of percentage glycosylated hemoglobin (HA1 and HA1c) as the standard for evaluation of blood glucose control (DCCT, N Engl J Med. (1993) 329:977-86). Alternatively, characterization of daily insulin needs, C-peptide levels/response, hypoglycemic episodes, and/or FPIR may be used as markers of B-cell function or to establish a therapeutic index (see Keymeulen et al., N Engl J Med. (2005) 352:2598-608; Herold et al., Diabetes (2005) 54:1763-9; U.S. Pat. Appl. Pub. No. 2004/0038867 A1; and Greenbaum et al., Diabetes (2001) 50:470-6, respectively). For example, FPIR is calculated as the sum of insulin values at 1 and 3 minutes post IGTT, which are performed according to Islet Cell Antibody Register User's Study protocols (see, e.g., Bingley et al., Diabetes (1996) 45:1720-8 and McCulloch et al., Diabetes Care (1993) 16:911-5).

In some embodiments, the individuals predisposed to develop T1D can be a non-diabetic subject who is a relative of a patient with T1D. In some embodiments, the non-diabetic subject has 2 or more diabetes-related autoantibodies selected from islet cell antibodies (ICA), insulin autoantibodies (IAA), and antibodies to glutamic acid decarboxylase (GAD), tyrosine phosphatase (IA-2/ICA512) or ZnT8.

In some embodiments, the non-diabetic subject has abnormal glucose tolerance on oral glucose tolerance test (OGTT). Abnormal glucose tolerance on OGTT is defined as a fasting glucose level of 110-125 mg/dL, or 2 hour plasma of ≥140 and <200 mg/dL, or an intervening glucose value at 30, 60, or 90 minutes on OGTT >200 mg/dL.

In some embodiments, the non-diabetic subject who will respond to the anti-CD3 antibody such as teplizumab does not have antibodies against ZnT8. In some embodiments, such non-diabetic subject is HLA-DR4+ and is not HLA-DR3+. In some embodiments, such non-diabetic subject who will respond to the anti-CD3 antibody such as teplizumab demonstrates an increase, following administration (e.g., after 1 month, after 2 months, after 3 months, or longer or shorter), in the frequency (or relative amount) of TIGIT+KLRG1+CD8+ T-cells (e.g., by flow cytometry) in peripheral blood mononuclear cells.

In some embodiments, the prophylactically effective amount comprises a 10 to 14-day course of subcutaneous (SC) injection or intravenous (IV) infusion of the anti-CD3 antibody such as teplizumab at 10-1000 micrograms/meter squared (μg/m²). In one example, the prophylactically effective amount comprises a 14-day course IV infusion of the anti-CD3 antibody such as teplizumab at 51 μg/m², 103 μg/m², 207 μg/m², and 413 μg/m², on days 0-3, respectively, and one dose of 826 μg/m² on each of days 4-13. In some embodiments, the prophylactically effective amount delays median time to clinical diagnosis of T1D by at least 50%, at least 80%, or at least 90%, or at least 12 months, at least 18 months, at least 24 months, at least 36 months, at least 48 months, or at least 60 months, or longer.

In some embodiments, the course of dosing with the anti-CD3 antibody such as teplizumab can be repeated at 2 month, 4 month, 6 month, 8 month, 9 month, 10 month, 12 month, 15 month, 18 month, 24 month, 30 month, or 36 month intervals. In some embodiments, efficacy of the treatment with the anti-CD3 antibody such as teplizumab is determined as described herein, or as is known in the art, at 2 months, 4 months, 6 months, 9 months, 12 months, 15 months, 18 months, 24 months, 30 months, or 36 months subsequent to the previous treatment.

In some embodiments, a subject is administered one or more unit doses of approximately 0.5-50 ug/kg, approximately 0.5-40 ug/kg, approximately 0.5-30 ug/kg, approximately 0.5-20 ug/kg, approximately 0.5-15 ug/kg, approximately 0.5-10 ug/kg, approximately 0.5-5 ug/kg, approximately 1-5 ug/kg, approximately 1-10 ug/kg, approximately 20-40 ug/kg, approximately 20-30 ug/kg, approximately 22-28 ug/kg or approximately 25-26 ug/kg of the anti-CD3 antibody such as teplizumab to prevent, treat or ameliorate one or more symptoms of T1D. In some embodiments, a subject is administered one or more unit doses of about 200 ug/kg, 178 ug/kg, 180 ug/kg, 128 ug/kg, 100 ug/kg, 95 ug/kg, 90 ug/kg, 85 ug/kg, 80 ug/kg, 75 ug/kg, 70 ug/kg, 65 ug/kg, 60 ug/kg, 55 ug/kg, 50 ug/kg, 45 ug/kg, 40 ug/kg, 35 ug/kg, 30 ug/kg, 26 ug/kg, 25 ug/kg, 20 ug/kg, 15 ug/kg, 13 ug/kg, 10 ug/kg, 6.5 ug/kg, 5 ug/kg, 3.2 ug/kg, 3 ug/kg, 2.5 ug/kg, 2 ug/kg, 1.6 ug/kg, 1.5 ug/kg, 1 ug/kg, 0.5 ug/kg, 0.25 ug/kg, 0.1 ug/kg, or 0.05 ug/kg of the anti-CD3 antibody such as teplizumab to prevent, treat or ameliorate one or more symptoms of T1D.

In some embodiments, a subject is administered one or more doses of the anti-CD3antibody such as teplizumab at about 5-1200 ug/m², preferably, 51-826 ug/m². In some embodiments, a subject is administered one or more unit doses of 1200 ug/m², 1150 ug/m², 1100 ug/m², 1050 ug/m², 1000 ug/m², 950 ug/m², 900 ug/m², 850 ug/m², 800 ug/m², 750 ug/m², 700 ug/m², 650 ug/m², 600 ug/m², 550 ug/m², 500 ug/m², 450 ug/m², 400 ug/m², 350 ug/m², 300 ug/m², 250 ug/m², 200 ug/m², 150 ug/m², 100 ug/m², 50 ug/m², 40 ug/m², 30 ug/m², 20 ug/m², 15 ug/m², 10 ug/m², or 5 ug/m² of the anti-CD3 antibody such as teplizumab to prevent, treat, slow the progression of, delay the onset of or ameliorate one or more symptoms of T1D.

In some embodiments, the subject is administered a treatment regimen comprising one or more doses of a prophylactically effective amount of the anti-CD3 antibody such as teplizumab, wherein the course of treatment is administered over 2 days, 3 days, 4 days, 5 days, 6 days, 7 days, 8 days, 9 days, 10 days, 11 days, 12 days, 13 days or 14 days. In some embodiments, the treatment regimen comprises administering doses of the prophylactically effective amount every day, every 2nd day, every 3rd day or every 4th day. In some embodiments, the treatment regimen comprises administering doses of the prophylactically effective amount on Monday, Tuesday, Wednesday, Thursday of a given week and not administering doses of the prophylactically effective amount on Friday, Saturday, and Sunday of the same week until 14 doses, 13 doses, 12 doses, 11 doses, 10 doses, 9 doses, or 8 doses have been administered. In some embodiments, the dose administered is the same each day of the regimen.

In some embodiments, a subject is administered a treatment regimen comprising one or more doses of a prophylactically effective amount of the anti-CD3 antibody such as teplizumab, wherein the prophylactically effective amount is 200 ug/kg/day, 175 ug/kg/day, 150 ug/kg/day, 125 ug/kg/day, 100 ug/kg/day, 95 ug/kg/day, 90 ug/kg/day, 85 ug/kg/day, 80 ug/kg/day, 75 ug/kg/day, 70 ug/kg/day, 65 ug/kg/day, 60 ug/kg/day, 55 ug/kg/day, 50 ug/kg/day, 45 ug/kg/day, 40 ug/kg/day, 35 ug/kg/day, 30 ug/kg/day, 26 ug/kg/day, 25 ug/kg/day, 20 ug/kg/day, 15 ug/kg/day, 13 ug/kg/day, 10 ug/kg/day, 6.5 ug/kg/day, 5 ug/kg/day, 3.2 ug/kg/day, 3 ug/kg/day, 2.5 ug/kg/day, 2 ug/kg/day, 1.6 ug/kg/day, 1.5 ug/kg/day, 1 ug/kg/day, 0.5 ug/kg/day, 0.25 ug/kg/day, 0.1 ug/kg/day, or 0.05 ug/kg/day; and/or wherein the prophylactically effective amount is 1200 ug/m²/day, 1150 ug/m²/day, 1100 ug/m²/day, 1050 ug/m²/day, 1000 ug/m²/day, 950 ug/m²/day, 900 ug/m²/day, 850 ug/m²/day, 800 ug/m²/day, 750 ug/m²/day, 700 ug/m²/day, 650 ug/m²/day, 600 ug/m²/day, 550 ug/m²/day, 500 ug/m²/day, 450 ug/m²/day, 400 ug/m²/day, 350 ug/m²/day, 300 ug/m²/day, 250 ug/m² day, 200 ug/m²/day, 150 ug/m²/day, 100 ug/m²/day, 50 ug/m²/day, 40 ug/m² day, 30 ug/m²/day, 20 ug/m²/day, 15 ug/m²/day, 10 ug/m²/day, or 5 ug/m²/day.

In some embodiments, the intravenous dose of 1200 ug/m² or less, 1150 ug/m² or less, 1100 ug/m² or less, 1050 ug/m² or less, 1000 ug/m² or less, 950 ug/m² or less, 900 ug/m² or less, 850 ug/m² or less, 800 ug/m² or less, 750 ug/m² or less, 700 ug/m² or less, 650 ug/m² or less, 600 ug/m² or less, 550 ug/m² or less, 500 ug/m² or less, 450 ug/m² or less, 400 ug/m² or less, 350 ug/m² or less, 300 ug/m² or less, 250 ug/m² or less, 200 ug/m² or less, 150 ug/m² or less, 100 ug/m² or less, 50 ug/m² or less, 40 ug/m² or less, 30 ug/m² or less, 20 ug/m² or less, 15 ug/m² or less, 10 ug/m² or less, or 5 ug/m² or less of the anti-CD3 antibody such as teplizumab is administered over about 24 hours, about 22 hours, about 20 hours, about 18 hours, about 16 hours, about 14 hours, about 12 hours, about 10 hours, about 8 hours, about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat or ameliorate one or more symptoms of type 1 diabetes. The total dosage over the duration of the regimen is preferably a total of less than 9000 ug/m², 8000 ug/m², 7000 ug/m², 6000 ug/m², and may be less than 5000 ug/m², 4000 ug/m², 3000 ug/m², 2000 ug/m², or 1000 ug/m². In some embodiments, the total dosage administered in the regimen is 100 ug/m² to 200 ug/m², 100 ug/m² to 500 ug/m², 100 ug/m² to 1000 ug/m², or 500 ug/m² to 1000 ug/m².

In some embodiments, the dose escalates over the first fourth, first half or first ⅔ of the doses (e.g., over the first 2, 3, 4, 5, or 6 days of a 10, 12, 14, 16, 18 or 20-day regimen of one dose per day) of the treatment regimen until the daily prophylactically effective amount of the anti-CD3 antibody such as teplizumab is achieved. In some embodiments, a subject is administered a treatment regimen comprising one or more doses of a prophylactically effective amount of the anti-CD3 antibody such as teplizumab, wherein the prophylactically effective amount is increased by, e.g., 0.01 ug/kg, 0.02 ug/kg, 0.04 ug/kg, 0.05 ug/kg, 0.06 ug/kg, 0.08 ug/kg, 0.1 ug/kg, 0.2 ug/kg, 0.25 ug/kg, 0.5 ug/kg, 0.75 ug/kg, 1 ug/kg, 1.5 ug/kg, 2 ug/kg, 4 ug/kg, 5 ug/kg, 10 ug/kg, 15 ug/kg, 20 ug/kg, 25 ug/kg, 30 ug/kg, 35 ug/kg, 40 ug/kg, 45 ug/kg, 50 ug/kg, 55 ug/kg, 60 ug/kg, 65 ug/kg, 70 ug/kg, 75 ug/kg, 80 ug/kg, 85 ug/kg, 90 ug/kg, 95 ug/kg, 100 ug/kg, or 125 ug/kg each day; or increased by, e.g., 1 ug/m², 5 ug/m², 10 ug/m², 15 ug/m², 20 ug/m², 30 ug/m², 40 ug/m², 50 ug/m², 60 ug/m², 70 ug/m², 80 ug/m², 90 ug/m², 100 ug/m², 150 ug/m², 200 ug/m², 250 ug/m², 300 ug/m², 350 ug/m², 400 ug/m², 450 ug/m², 500 ug/m², 550 ug/m², 600 ug/m², or 650 ug/m², each day as treatment progresses. In some embodiments, a subject is administered a treatment regimen comprising one or more doses of a prophylactically effective amount of the anti-CD3 antibody such as teplizumab, wherein the prophylactically effective amount is increased by a factor of 1.25, a factor of 1.5, a factor of 2, a factor of 2.25, a factor of 2.5, or a factor of 5 until the daily prophylactically effective amount of the anti-CD3 antibody such as teplizumab is achieved.

In some embodiments, a subject is intramuscularly administered one or more doses of a 200 ug/kg or less, preferably 175 ug/kg or less, 150 ug/kg or less, 125 ug/kg or less, 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, to prevent, treat or ameliorate one or more symptoms of T1D.

In some embodiments, a subject is subcutaneously administered one or more doses of a 200 ug/kg or less, preferably 175 ug/kg or less, 150 ug/kg or less, 125 ug/kg or less, 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, to prevent, treat or ameliorate one or more symptoms of T1D.

In some embodiments, a subject is intravenously administered one or more doses of a 100 ug/kg or less, preferably 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, to prevent, treat or ameliorate one or more symptoms of T1D. In some embodiments, the intravenous dose of 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat or ameliorate one or more symptoms of T1D.

In some embodiments, a subject is orally administered one or more doses of a 100 ug/kg or less, preferably 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, I ug/kg or less, 0.5 ug/kg or less, or 0.2 ug/kg or less of the anti-CD3 antibody such as teplizumab, otelixizumab or foralumab, to prevent, treat or ameliorate one or more symptoms of T1D. In some embodiments, the oral dose of 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat or ameliorate one or more symptoms of T1D.

In some embodiments in which escalating doses are administered for the first days of the dosing regimen, the dose on day 1 of the regimen is 5-100 ug/m²/day, preferably 51 ug/m²/day and escalates to the daily dose as recited immediately above by day 3, 4, 5, 6 or 7. For example, on day 1, the subject is administered a dose of approximately 51 ug/m²/day, on day 2 approximately 103 ug/m²/day, on day 3 approximately 207 ug/m²/day, on day 4 approximately 413 ug/m²/day and on subsequent days of the regimen (e.g., days 5-14) 826 ug/m²/day. In some embodiments, on day 1, the subject is administered a dose of approximately 227 ug/m²/day, on day 2 approximately 459 ug/m²/day, on day 3 and subsequent days, approximately 919 ug/m²/day. In some embodiments, on day 1, the subject is administered a dose of approximately 284 ug/m²/day, on day 2 approximately 574 ug/m²/day, on day 3 and subsequent days, approximately 1148 ug/m²/day.

In some embodiments, the initial dose is ¼, to ½, to equal to the daily dose at the end of the regimen but is administered in portions at intervals of 6, 8, 10 or 12 hours. For example, a 13 ug/kg/day dose is administered in four doses of 3-4 ug/kg at intervals of 6 hours to reduce the level of cytokine release caused by administration of the antibody. In some embodiments, to reduce the possibility of cytokine release and other adverse effects, the first 1, 2, 3, or 4 doses or all the doses in the regimen are administered more slowly by intravenous administration. For example, a dose of 51 ug/m²/day may be administered over about 5 minutes, about 15 minutes, about 30 minutes, about 45 minutes, about 1 hour, about 2 hours, about 4 hours, about 6 hours, about 8 hours, about 10 hours, about 12 hours, about 14 hours, about 16 hours, about 18 hours, about 20 hours, and about 22 hours. In some embodiments, the dose is administered by slow infusion over a period of, e.g., 20 to 24 hours. In some embodiments, the dose is infused in a pump, preferably increasing the concentration of antibody administered as the infusion progresses.

In some embodiments, a set fraction of the doses for the 51 ug/m²/day to 826 ug/m²/day regimen described above is administered in escalating doses. In some embodiments, the fraction is 1/10, ¼, ⅓, ½, ⅔ or ¾ of the daily doses of the regimens described above. Accordingly, when the fraction is 1/10, the daily doses will be 5.1 ug/m² on day 1, 10.3 ug/m² on day 2, 20.7 g/m² on day 3, 41.3 ug/m² on day 4, and 82.6 ug/m² on days 5 to 14. When the fraction is ¼, the doses will be 12.75 ug/m² on day 1, 25.5 ug/m² on day 2, 51 ug/m² on day 3, 103 ug/m² on day 4, and 207 ug/m² on days 5 to 14. When the fraction is ⅓, the doses will be 17 ug/m² on day 1, 34.3 ug/m² on day 2, 69 ug/m² on day 3, 137.6 ug/m² on day 4, and 275.3 ug/m² on days 5 to 14. When the fraction is ½, the doses will be 25.5 ug/m² on day 1, 51 ug/m² on day 2, 103 ug/m² on day 3, 207 ug/m² on day 4, and 413 ug/m² on days 5 to 14. When the fraction is ⅔, the doses will be 34 ug/m² on day 1, 69 ug/m² on day 2, 137.6 ug/m² on day 3, 275.3 ug/m² on day 4, and 550.1 ug/m² on days 5 to 14. When the fraction is ¾, the doses will be 38.3 ug/m² on day 1, 77.3 ug/m² on day 2, 155.3 ug/m² on day 3, 309.8 ug/m² on day 4, and 620 ug/m² on days 5 to 14. In some embodiments, the regimen is identical to one of those described above but only over days 1 to 4, days 1 to 5, or days 1 to 6. For example, in some embodiments, the doses will be 17 ug/m² on day 1, 34.3 ug/m² on day 2, 69 ug/m² on day 3, 137.6 ug/m² on day 4, and 275.3 ug/m² on days 5 and 6.

In some embodiments, the anti-CD3 antibody such as teplizumab, otelixizumab or foralumab, is not administered by daily doses over a number of days, but is rather administered by infusion in an uninterrupted manner over 4 hours, 6 hours, 8 hours, 10 hours, 12 hours, 15 hours, 18 hours, 20 hours, 24 hours, 30 hours or 36 hours. The infusion may be constant or may start out at a lower dosage for, for example, the first 1, 2, 3, 5, 6, or 8 hours of the infusion and then increase to a higher dosage thereafter. Over the course of the infusion, the patient receives a dose equal to the amount administered in the 5 to 20-day regimens set forth above. For example, a dose of approximately 150 ug/m², 200 ug/m², 250 ug/m², 500 ug/m², 750 ug/m², 1000 ug/m², 1500 ug/m², 2000 ug/m², 3000 ug/m², 4000 ug/m², 5000 ug/m², 6000 ug/m², 7000 ug/m², 8000 ug/m², or 9000 ug/m². In particular, the speed and duration of the infusion is designed to minimize the level of free anti-CD3 antibody such as teplizumab, otelixizumab or foralumab in the subject after administration. In some embodiments, the level of free anti-CD3 antibody such as teplizumab should not exceed 200 ng/ml free antibody. In addition, the infusion is designed to achieve a combined T cell receptor coating and modulation of at least 50%, 60%, 70%, 80%, 90%, 95% or of 100%.

In some embodiments, the anti-CD3 antibody such as teplizumab, otelixizumab or foralumab is administered chronically to treat, prevent, or slow or delay the onset or progression, or ameliorate one or more symptoms of type 1 diabetes. For example, in some embodiments, a low dose of the anti-CD3 antibody such as teplizumab is administered once a month, twice a month, three times per month, once a week or even more frequently either as an alternative to the 6 to 14-day dosage regimen discussed above or after administration of such a regimen to enhance or maintain its effect. Such a low dose may be anywhere from 1 ug/m² to 100 ug/m², such as approximately 5 ug/m², 10 ug/m², 15 ug/m², 20 ug/m², 25 ug/m², 30 ug/m², 35 ug/m², 40 ug/m², 45 ug/m², or 50 ug/m².

In some embodiments, the subject may be re-dosed at some time subsequent to administration of the anti-CD3 antibody such as teplizumab, otelixizumab or foralumab dosing regimen, for example, based upon one or more physiological parameters or may be done as a matter of course. Such redosing may be administered and/or the need for such redosing evaluated 2 months, 4 months, 6 months, 8 months, 9 months, 1 year, 15 months, 18 months, 2 years, 30 months or 3 years after administration of a dosing regimen and may include administering a course of treatment every 6 months, 9 months, 1 year, 15 months, 18 months, 2 years, 30 months or 3 years indefinitely.

### EXAMPLE

### Example 1: Teplizumab Improves and Stabilizes Beta Cell Function in Antibody Positive High-Risk Individuals

### Abstract

We analyzed the effects of a single 14-day course of teplizumab treatment on metabolic function and immune cells among participants in a randomized controlled trial of non-diabetic relatives at high-risk for Type 1 diabetes (T1D). In an extended follow up (923-day median) of a previous report of teplizumab treatment the median times to diagnosis were 59.6 and 24.4 (or 27.1 according to Sims et al., Sci Transl Med. (2021) 13:eabc8980, incorporated herein by reference) months for teplizumab and placebo treated participants, respectively (HR=0.457, p=0.01). Fifty percent of teplizumab-treated but only 22% of the placebo-treated remained diabetes free. Glucose tolerance, C-peptide area under the curve (AUC), and insulin secretory rates were calculated, and relationships to T cell subsets and function were analyzed. Teplizumab treatment improved beta cell function, reflected by average on-study C-peptide AUC (1.96 vs 1.68 pmol/ml; p=0.009) (or 1.94 versus 1.72 pmol/ml; P=0.006 according to Sims et al., Sci Transl Med. (2021) 13:eabc8980, incorporated herein by reference).

Drug treatment reversed a decline in insulin secretion prior to enrollment followed by stabilization of the declining C-peptide AUC seen with placebo treatment. The changes in C-peptide with teplizumab treatment were associated with increases in partially exhausted memory KLRG1+TIGIT+CD8+ T-cells (r=0.44; p=0.014) and reduced secretion of IFNγ and TNFα. A single course of teplizumab had lasting effects on delay of T1D diagnosis and improved beta cell function in high-risk individuals. Changes in CD8+ T-cell subsets indicate that partially exhausted effector cells are associated with clinical response. This is the first trial to show successful modulation of autoimmune diabetes with immune therapy.

### Introduction

Type 1 diabetes (T1D) is an autoimmune disease characterized by T-cell mediated destruction of insulin producing beta cells within the pancreatic islets of Langerhans. Longitudinal observational studies over more than 30 years have described the progression of the autoimmune disease from the first appearance of autoantibodies until beta cell function is critically impaired and the clinical diagnosis, often with ketoacidosis, occurs (1-5). T1D is associated with a need for lifelong exogenous insulin administration for survival, increased morbidity and mortality due to immediate (e.g., hypoglycemia) and long-term complications (e.g., vascular, renal, and eye disease), and reduced life-span, life impairments, and considerable health-care-related costs (6-9). Thus, approaches to prevent progression to clinical T1D before irremediable beta cell destruction and insulin deficiency, are of paramount importance.

Changes in beta cell function precede the clinical diagnosis of T1D and have been studied in natural history cohorts of individuals who are identified as at-risk for the disease based on the presence of islet autoantibodies (10-12). Some studies suggest an ongoing and intermittently progressive decline in beta cell function, that begins years before clinical diagnosis at a time when glucose tolerance is normal. During this period there are signs of ongoing autoimmunity: Based on the findings of the natural history, individuals with two or more islet autoantibodies have been classified as stages of T1D, with further specification according to the level of metabolic dysfunction: Stage 1 prior to glucose abnormalities, Stage 2 with dysglycemia during an oral glucose tolerance test (OGTT), and Stage 3 at clinical presentation with hyperglycemia (2, 13, 14). However, the relationships between changes in beta cell function and clinical disease remain poorly defined. It is known, for example, that glucose tolerance, defined through responses to an oral glucose tolerance test (OGTT), may fluctuate between abnormal and normal values within an individual who is at risk (15, 16). In addition, OGTT glucose tolerance classifications used to designate a clinical diagnosis, and beta cell function, measured by C-peptide responses to a metabolic challenge, may not be closely related and at diagnosis using an OGTT, many individuals have clinically meaningful C-peptide responses (15-18). Based on successes from previous studies in patients with Stage 3 T1D (i.e., after clinical diagnosis) with teplizumab, an Fc receptor-nonbinding anti-CD3ε monoclonal antibody, that showed reduced decline in stimulated C-peptide responses compared to placebo or control participants (19-25), in the TrialNet TN10 study, we conducted a randomized Phase II trial of teplizumab in individuals with Stage 2 disease, to test whether treatment would prevent or delay the clinical diagnosis of T1D (26). In this time-to-event study, we found a delay in the median time to diagnosis of 24 months with teplizumab vs placebo, and a reduction in the rate of diabetes diagnoses from 35.9% to 14.9% per year (26). This trial represented the first to show successful prevention or delay in the diagnosis of T1D with immune therapy (27-31).

The successful outcome of the TrialNet TN10, using an intervention that modified clinical disease, has enabled us to evaluate the effects of the therapy on beta cell function and its relationship to immune modifications, even when disease progression was clinically silent. To test the hypothesis that the immune therapy would improve beta cell function in the at-risk individuals from TN10, we analyzed the results of metabolic studies in the trial and immune responses. Our data show a persistence of the duration of treatment effects on delay in clinical presentation of T1D. We show that a single course of treatment with teplizumab reversed a decline in C-peptide production prior to study entry and improved the beta cell response to oral glucose after treatment compared to placebo. Early insulin secretion was also improved with teplizumab, suggesting qualitative improvement in beta cell function. After the initial 3-6 months following treatment, the C-peptide responses were stable compared to placebo until an abrupt decline in the response approximately 6 months prior to diagnosis in those who were diagnosed with clinical T1D. The improved C-peptide responses were associated with an increase in the frequency of TIGIT+KLRG1+ memory CD8 T-cells, which exhibited reduced secretion of IFNγ and TNFα, two inflammatory cytokines linked to beta cell destruction (32). These studies indicate that even prior to clinical diagnosis, treatment with teplizumab can improve metabolic function associated with modulation of pathologic T cell signatures.

### Results

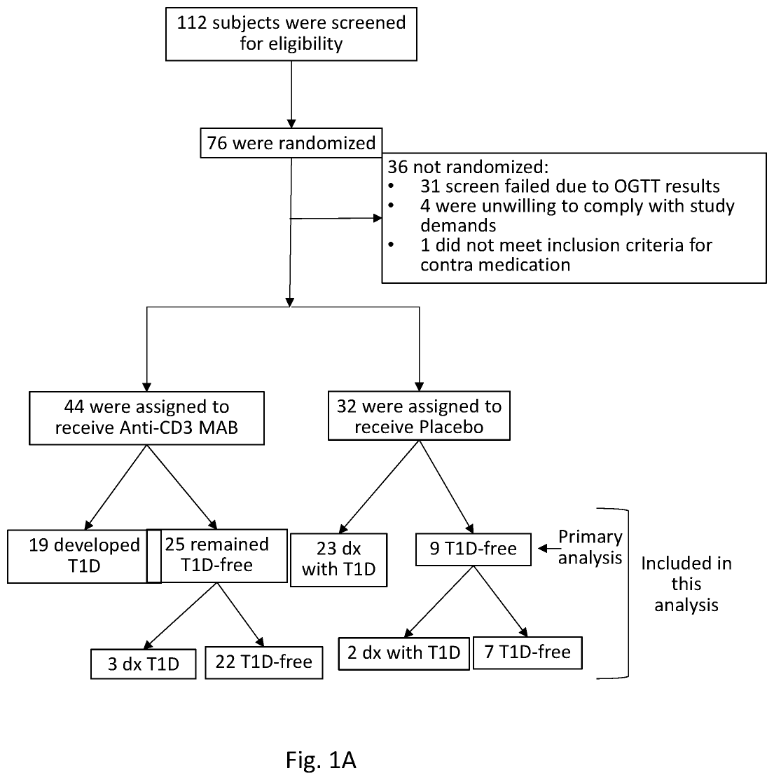

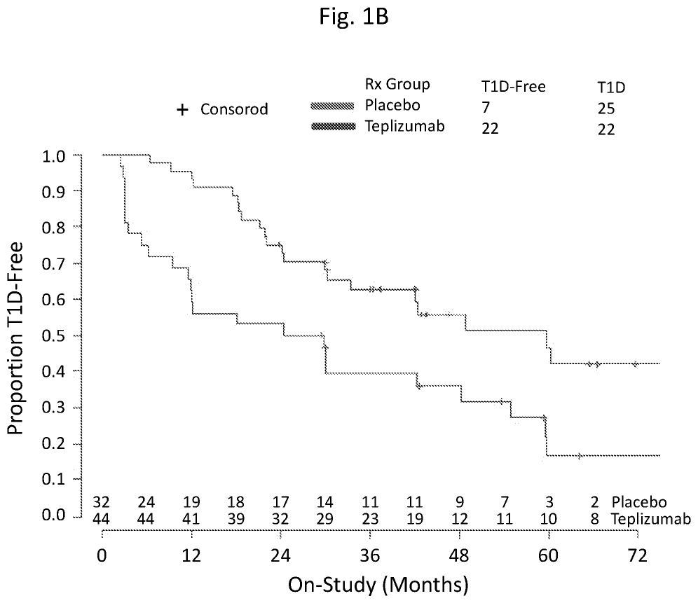

Teplizumab treatment resulted in a sustained delay in T1D during extended follow-up studies: A total of 76 relatives at high-risk, but without a clinical diagnosis of T1D were enrolled into the teplizumab prevention study (26). The median age was 13 (range 8-49) and all participants had 2+ autoantibody tests within the 6 months prior to enrollment. We previously reported, after a median follow up of 742 days (range 74 to 2683) that 42 were diagnosed with T1D. We have since continued to follow the study participants for a median time of 923 days (range of 74-3,119) (FIG. 1A). Over this extended period of follow-up, 25/32 (78%) of the placebo-treated and 22/44 (50%) of the teplizumab-treated participants were diagnosed with T1D (FIG. 1B) (Cox model adjusting for stratification and age: HR=0.457 p=0.01). The median times to diagnosis of T1D were 59.6 and 24.4 (or 27.1 according to Sims et al., Sci Transl Med. (2021) 13:eabc8980, incorporated herein by reference) months in the teplizumab and placebo treatment groups respectively. Ten of thirteen subjects followed beyond 60 months or 5 years were not diagnosed with T1D. Of these individuals, eight were in the teplizumab group and two were in the placebo group.

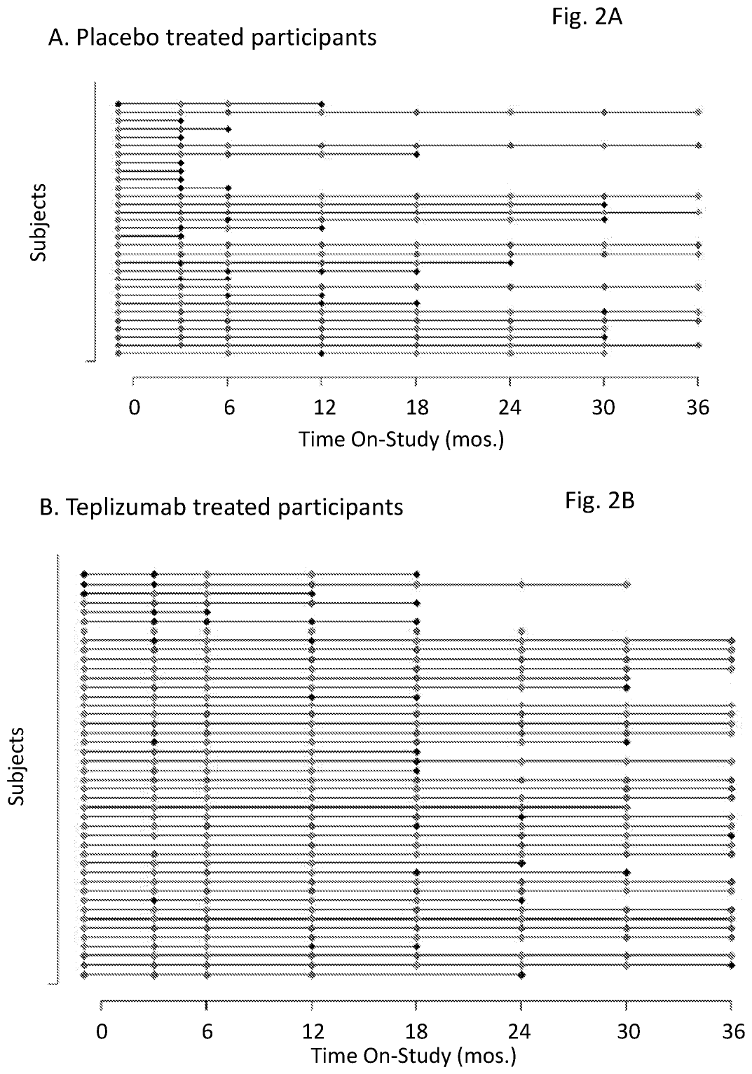

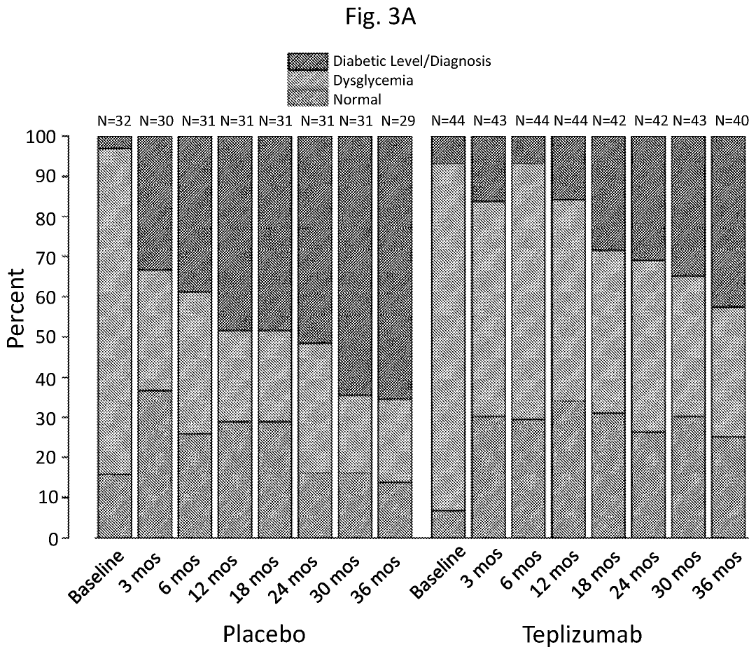

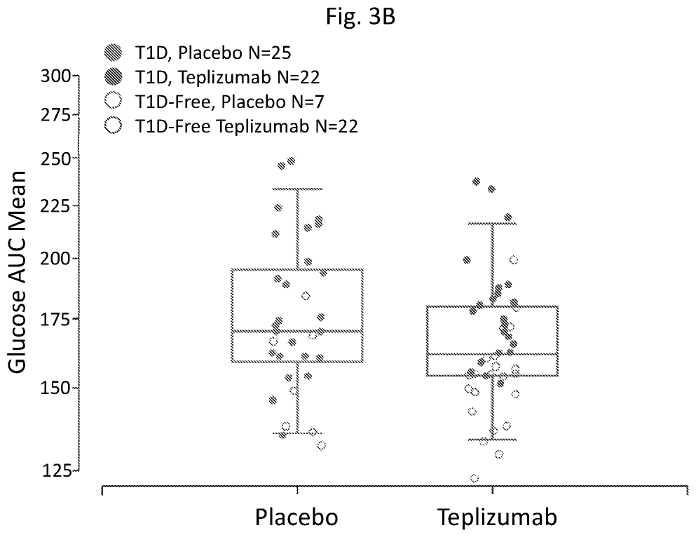

Teplizumab treatment improved quantitative OGTT glucose AUC values over the course of the study: To determine how teplizumab treatment affected glucose tolerance, we classified the outcomes of the OGTTs as normal, dysglycemic, or diabetic at study entry and tallied the frequency of these outcomes at each study visit over the first 36 months of study and afterwards (FIGS. 2A and 2B; FIG. 3A). Study participants had been recruited on the basis of a dysglycemic OGTT test result. At randomization, and consistent with the known variability of OGTT results, a small number of subjects had normal (n=3) or diabetic (n=6) glucose tolerance at that visit. The clinical diagnosis of T1D, the primary endpoint of the study, required two consecutive diabetic OGTTs hence, participants could continue in the study with a single diabetic OGTT. At the 3 month visit after teplizumab or placebo administration, the frequency of dysglycemic OGTTs declined and the frequency of normal OGTTs increased in both groups (in teplizumab treated from 6.8% to 30.2%, McNemar test: p=0.009; and in placebo from 15.6% to 36.7%, McNemar test: p=0.02). Diabetic OGTTs also increased in both groups at this timepoint, particularly in the placebo group. Afterwards, the frequency of normal and dysglycemic OGTTs remained relatively constant in the teplizumab group: the frequency of diabetic OGTTs increased in both groups but at a slower rate in the teplizumab treated participants.

Changes in OGTT classifications could overlook more subtle effects of treatment on the OGTT glucose responses. We therefore calculated and compared an average on-study glucose AUC for each individual, which was corrected for the time in study. The average on-study glucose AUC was higher in those treated with placebo vs teplizumab (mean (IQR) 175 (159, 195) mg/dl vs 165 (154, 180) mg/dl, ANCOVA teplizumab effect: 92.8%, p=0.02). (FIG. 3B, Table 1). The individual glucose AUC at the time of study entry was a predictor of the average on-study glucose AUC, but values at entry were similar between groups (unadjusted group geometric means of placebo and teplizumab: 155.5 mg/dl for placebo and 162.2 mg/dl for teplizumab, p=0.25).

TABLE 1
ANCOVA Model of Glucose On-study AUC Mean (Ln transform)

| Covariate | Coefficient | Standard Error | t-test | p-value |
|---|---|---|---|---|
| (Intercept) | 2.96 | 0.602 | 4.91 | <0.0001 |
| Glucose | 0.44 | 0.119 | 3.7 | 0.0004 |
| (baseline) | | | | |
| Age | −0.000383 | 0.00145 | −0.264 | 0.79 |
| Teplizumab | −0.0804* | 0.0343 | −2.35 | 0.02 |
| treatment | | | | |

*Mean of teplizumab group is 92.8% of the placebo group

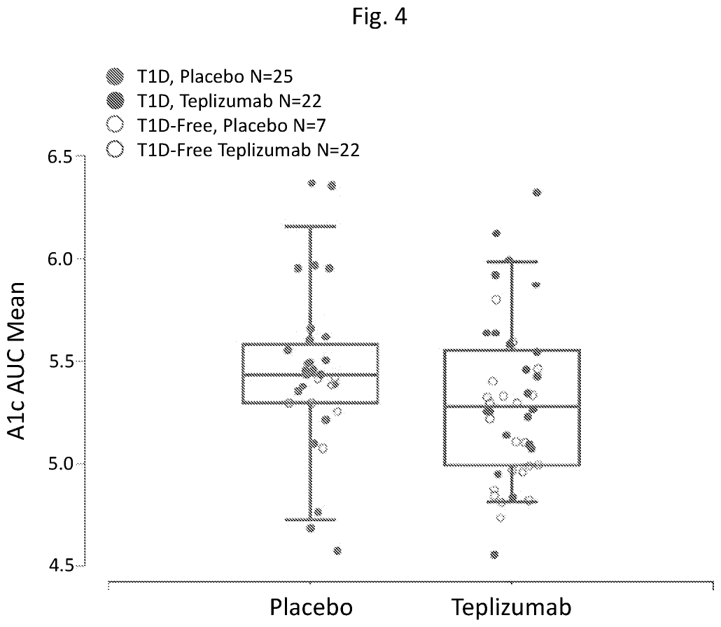

Average on-study Hemoglobin A1c (HbA1c) AUC was also calculated and analyzed. In contrast to glucose, the average on-study HbA1c AUC was not statistically different in those treated with placebo vs teplizumab (mean (IQR) 5.44% (5.29, 5.58) vs 5.3% (4.99, 5.55), ANCOVA treatment: p=0.14) (FIG. 4). Because the frequency of diabetes was higher in the placebo group, the similarity in the HbA1c, a measure of chronic glucose exposure, the higher average on-study glucose AUC level in the placebo group most likely was due to acute rather than chronic changes in glucose levels.

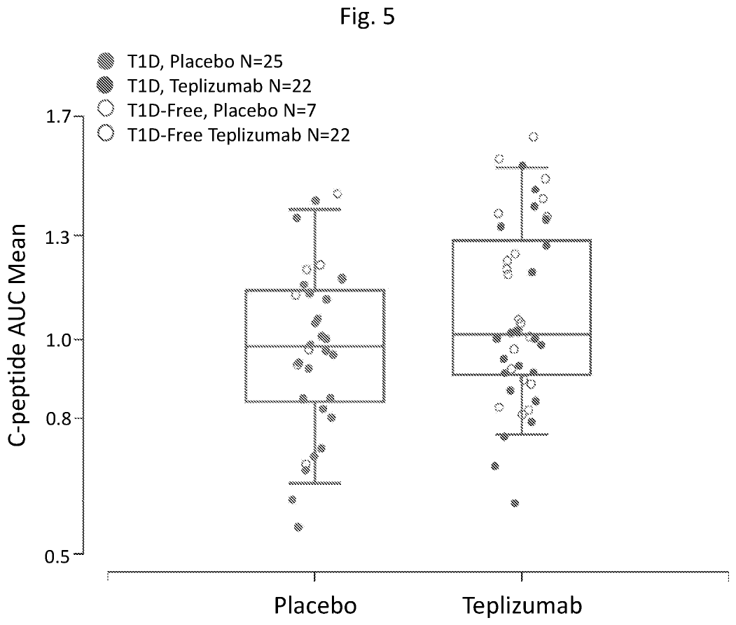

Teplizumab treatment increased C-peptide responses: The average on-study C-peptide AUC was greater in the teplizumab treatment group vs placebo (mean (IQR) 1.96 (1.48, 2.61) pmol/ml vs 1.68 (1.32, 2.11) pmol/ml (p=0.009) (FIG. 5, Table 2) (or 1.94 versus 1.72 pmol/ml; P=0.006 according to Sims et al., Sci. Transl. Med. 13, eabc8980 (2021), incorporated herein by reference). To assess the relationship of this endpoint with diabetes development, we compared average on-study C-peptide AUC values between participants who did or did not develop T1D during the period of observation. For the entire study population, average on-study C-peptide AUC was greater in individuals that remained diabetes free compared to those that progressed to T1D (mean (IQR) 2.18 (1.52, 2.79) pmol/ml vs. 1.76 (1.30, 2.18) pmol/ml (p=0.016). However, there was not a clear difference in the average C-peptide levels in those who were diagnosed and remained diabetes free within each treatment arm. In the placebo arm, 11/17 and 10/16 individuals in the lower and upper half of individuals respectively were diagnosed with T1D and in the teplizumab arm 13/22 and 7/22 in the lower and upper half of individuals were diagnosed with T1D (Chi-squared p=0.13) (FIG. 5).

TABLE 2
ANCOVA Model of A1c On-study AUC Mean (Ln-Ln transform)

| Covariate | Coefficient | Standard Error | t-test | p-value |
|---|---|---|---|---|
| (Intercept) | 0.0971 | 0.0481 | 2.02 | 0.05 |
| A1c (baseline) | 0.866 | 0.0964 | 8.98 | <0.0001 |
| Age | −0.000231 | 0.0003.6 | −0.755 | 0.45 |
| Teplizumab | −0.0107 | 0.00712 | −1.5 | 0.14 |
| treatment | | | | |

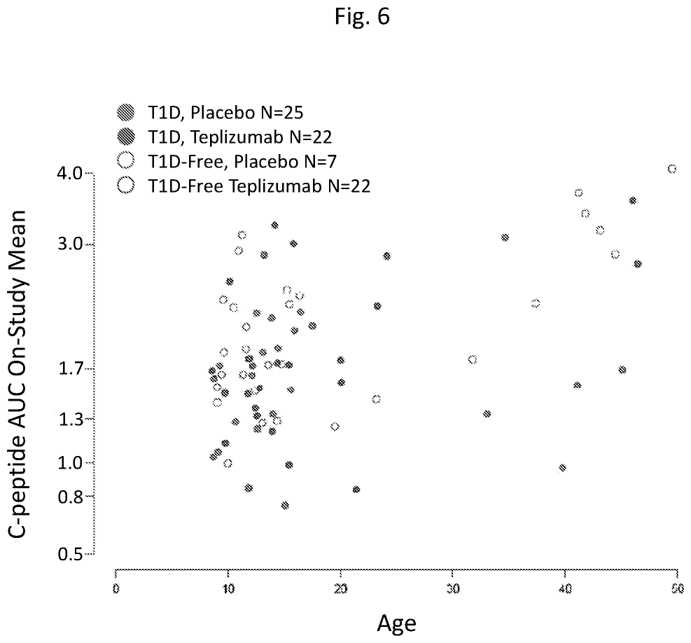

Baseline C-peptide AUC (p<0.0001) was a significant determinant of the average on-study C-peptide AUC, but baseline values were similar between treatment groups (unadjusted group means for placebo and teplizumab of 1.95 pmol/ml and 1.99 pmol/ml (p=0.454). There was also a direct relationship between the participant age and the average C-peptide AUC across both treatment arms and outcomes (FIG. 6) (from ANCOVA, r=0.44, p=0.0001) as has been noted previously in studies of individuals at risk and with new onset T1D (33). In contrast to relationships with time to clinical diagnosis (26), HLA-DR4+, HLA-DR3−, or anti-ZnT8 antibody status did not show significant interactions with the average on-study C-peptide AUC. (Using Wald test: HLA-DR3 p=0.71, HLA-DR4 p=0.27, ZnT8 p=0.79).

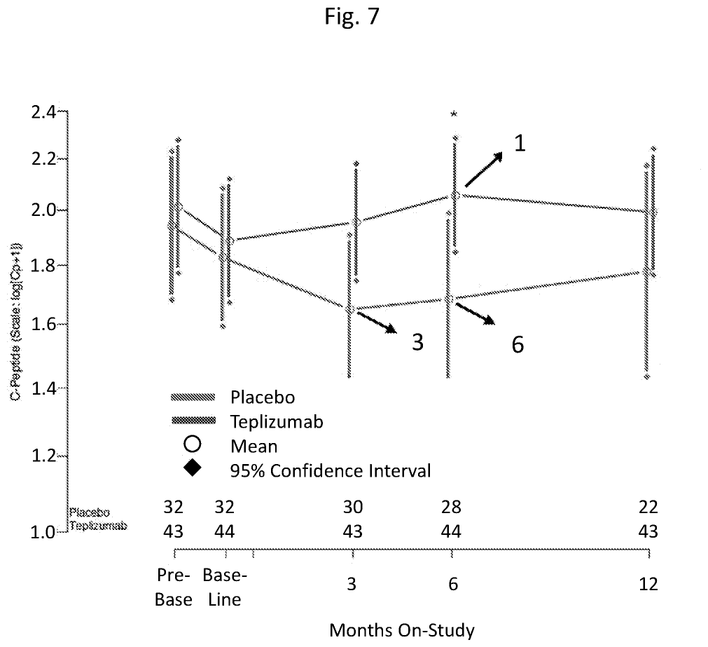

Teplizumab treatment reverses declines in C-peptide AUC during the first 6 months of treatment: Because average on-study C-peptide AUC could obscure more pronounced between-group differences at individual study timepoints, we next analyzed the timing of the changes in C-peptide AUC relative to treatment and the insulin secretion patterns. As the participants had been recruited from the TN01 Natural History study, we were able to analyze the C-peptide response to OGTTs before enrollment, and compare these to values after enrollment in this study. Geometric-like group means over a median of 2.4 months prior to randomization, and over the 12 months after are shown in FIG. 7 and Tables 3-4. There was a decline in the C-peptide AUC prior to study enrollment in both groups (pre-baseline and baseline): placebo 1.94 (1.68, 2.23), and 1.83 pmol/ml (1.59, 2.08)), teplizumab: 2.01 (1.77, 2.28) and 1.89 pmol/ml (1.67, 2.12) with an average slope of −0.0202 (−0.0471, 0.0201) (n=43). In the participants treated with placebo, the decline in C-peptide persisted at the same rate for the first 6 months after enrollment (the mean C-peptide AUC of 1.62 pmol/ml (1.35, 1.91) over the 6 months), with no significant differences in pre vs. post treatment slopes even after correction for age and the C-peptide at enrollment. In contrast, there was a significant increase in the C-peptide AUC in the teplizumab treated participants at 6 months after enrollment (6 month mean C-peptide AUC of 2.06 pmol/ml (1.85, 2.29), paired t-test p=0.02). The post-treatment slopes differed significantly between the placebo and teplizumab treated participants by ANCOVA after correcting for age and the pre-treatment slope (p=0.002).

TABLE 3
ANCOVA Model of C-peptide On-study AUC Mean (In (x + 1) transform)

| Covariate | Coefficient | Standard Error | t-test | p-value |
|---|---|---|---|---|
| (Intercept) | −0.503 | 0.108 | −4.64 | <0.0001 |
| C-peptide | 2.67 | 0.209 | 12.8 | <0.0001 |
| (baseline) | | | | |
| Age | 0.00389 | 0.00129 | 3.02 | 0.003 |
| Teplizumab | 0.0756* | 0.0283 | 2.67 | 0.009 |
| treatment | | | | |

*predicted means for teplizumab and Placebo groups are 1.93 and 1.72 nano-moles/L respectively (baseline C-peptide and age set to the cohort mean)

TABLE 4
ANCOVA analysis of C-peptide AUC slope Over 1st 6 months on Study

| Covariate | Coefficient | Standard Error | t-test | p-value |
|---|---|---|---|---|
| Intercept | −0.0237 | 0.00717 | −3.3 | 0.002 |
| Pre-slope | −0.0801 | 0.0439 | −1.82 | 0.07 |
| Age | 0.00048 | 0.000284 | 1.69 | 0.10 |
| Teplizumab | 0.0212 | 0.00663 | 3.2 | 0.002 |
| treatment | | | | |

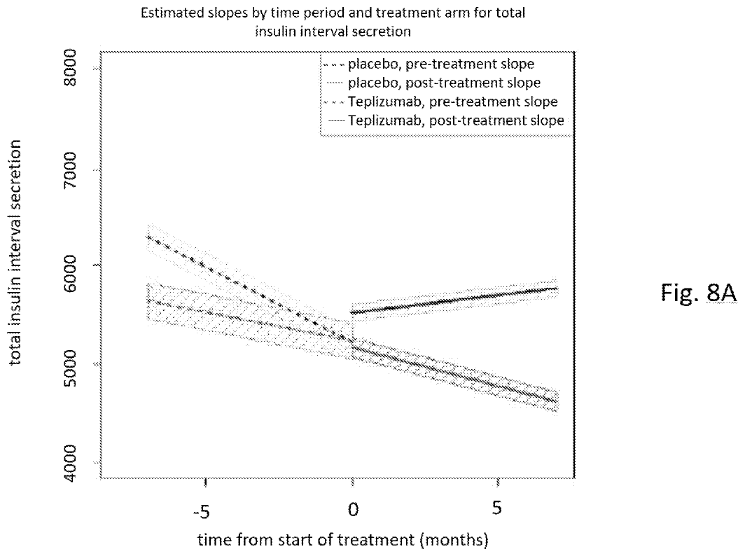

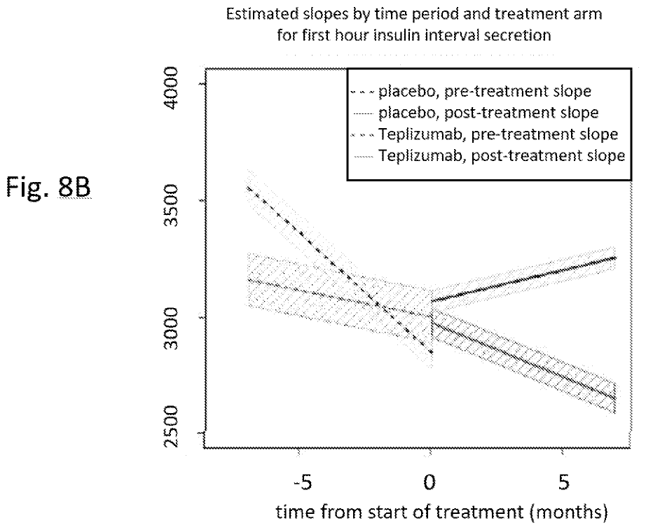

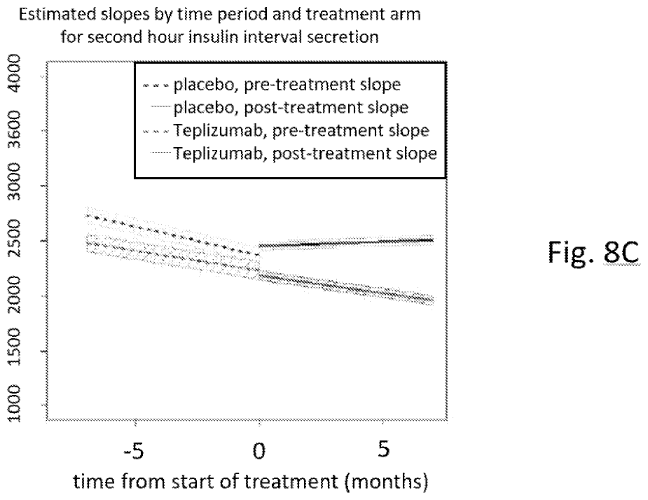

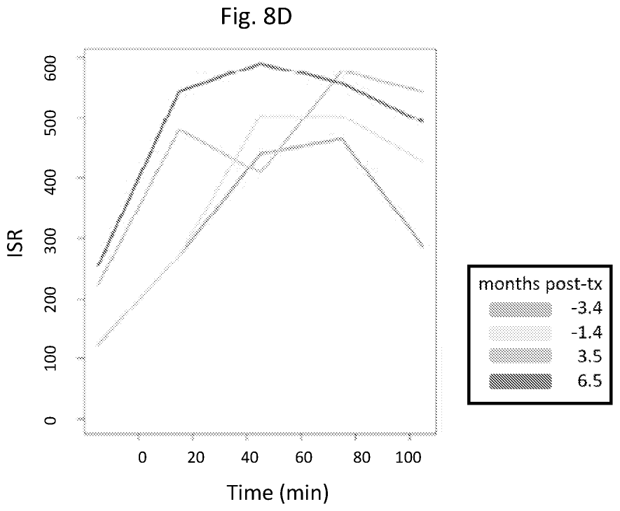

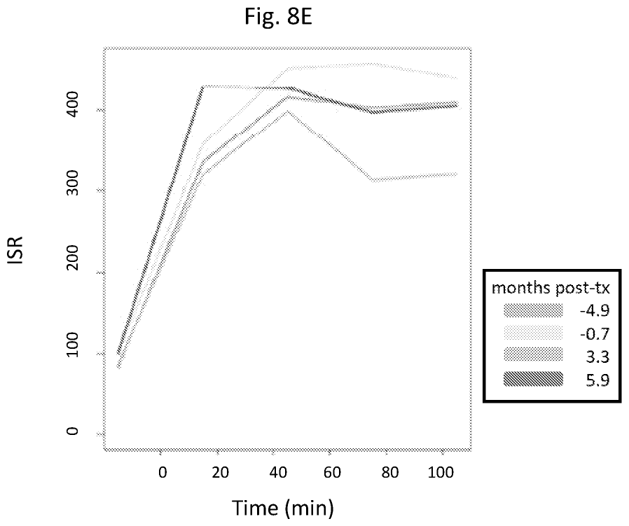

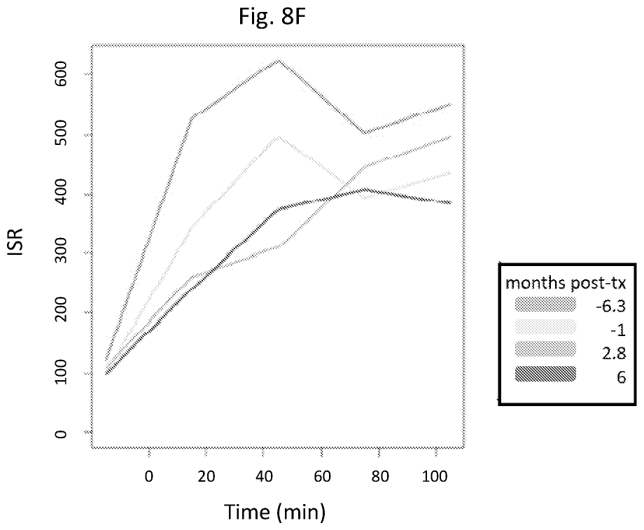

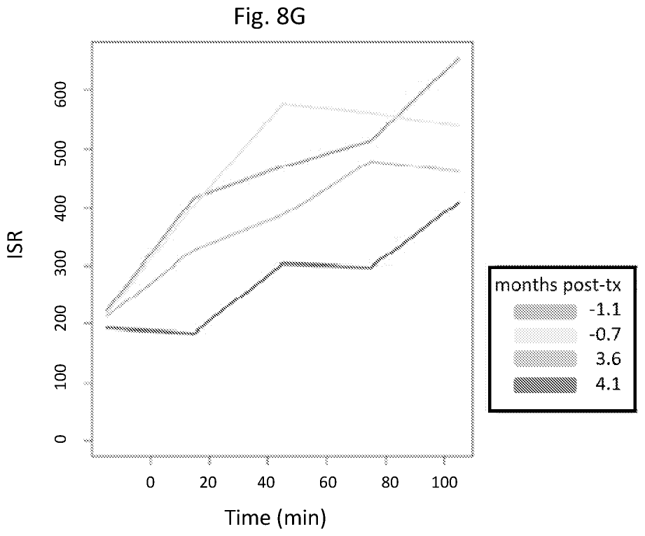

Both total and early insulin secretion are improved by teplizumab treatment: In addition to quantitative decreases in C-peptide AUC, studies by our group and others have identified qualitative abnormalities in beta cell secretory kinetics, with loss of early insulin secretion reflecting beta cell dysfunction prior to the onset of T1D (10, 33-36). To determine whether the quantitative improvement in C-peptide AUC was associated with qualitative changes in the kinetics of insulin secretion, we determined the insulin secretory rates (ISR) during the OGTTs using a two-compartment model, and evaluated the kinetics and total insulin secretion (FIGS. 8A-8G, Table 5). We compared the OGTT insulin secretory responses and the change in the secretory responses (slope) over the same period of time in which we had found a significant improvement in the C-peptide AUC in the teplizumab treatment arm. With this analysis we could distinguish the early and late secretory responses (i.e. first and second hour). The slopes describing the change in the total, first, and second hour insulin secreted was similar in both groups prior to study enrollment (p=0.95). After treatment with teplizumab there was a significant increase in the total insulin secreted during the test in the teplizumab group that was significantly greater than in the placebo group (p=0.01, p=0.0004). The insulin secreted during the first hour continued to decline in the placebo group whereas it increased in the teplizumab group (p=0.007). The second hour of insulin secretion also improved in the teplizumab treatment group (p=0.03), but not in the placebo group (p=0.38) (Table 5). These results indicate that in the first 6 months after teplizumab treatment there is improvement in insulin secretion, particularly within the first hour of the OGTT, suggesting improved beta cell function, whereas there is continuing deterioration in insulin secretion in the placebo treated participants.

TABLE 5
Analysis of insulin secretion to oral glucose in the first 6 months after treatment

Medians

| Measure to be compared between arms | Placebo | Teplizumab | p-value |
|---|---|---|---|
| First hour insulin interval secretion | | | |
| Pre-treatment slope | −259.5 | −422.7 | 0.79 |
| Post-treatment slope | −476.2 | 371.0 | 0.0003 |
| Paired pre- vs. post-rx | p = 0.86 | p = 0.007 | — |
| p-values * within arms | | | |
| Second hour insulin interval secretion | | | |
| Pre-treatment slope | −728.2 | −383.6 | 0.78 |
| Post-treatment slope | −186.8 | 442.5 | 0.003 |
| Paired pre- vs. post-rx | p = 0.38 | p = 0.03 | — |
| p-values * within arms | | | |
| Insulin interval secretion (2 hr) | | | |
| Pre-treatment slope | −1245.0 | −1024.0 | 0.95 |
| Post-treatment slope | −1037.4 | 1085.8 | 0.0004 |
| Paired pre- vs. post-rx | p = 0.80 | p = 0.01 | — |
| p-values* within arms | | | |

where diff = (post-rx slope) − (pre-rx slope)
*p-values based on Wilcoxon signed rank (paired) test comparing the pre- vs. post-rx slopes by subject; evaluation of how much changes in these measures changed pre- vs. post-rx by subject and across those subjects in each treatment arm.

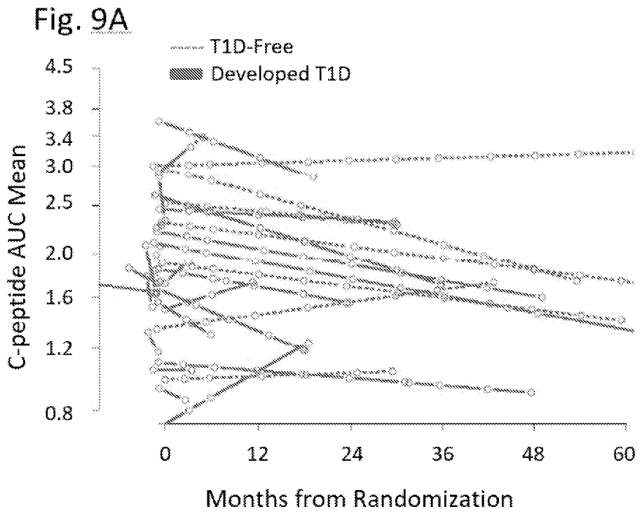

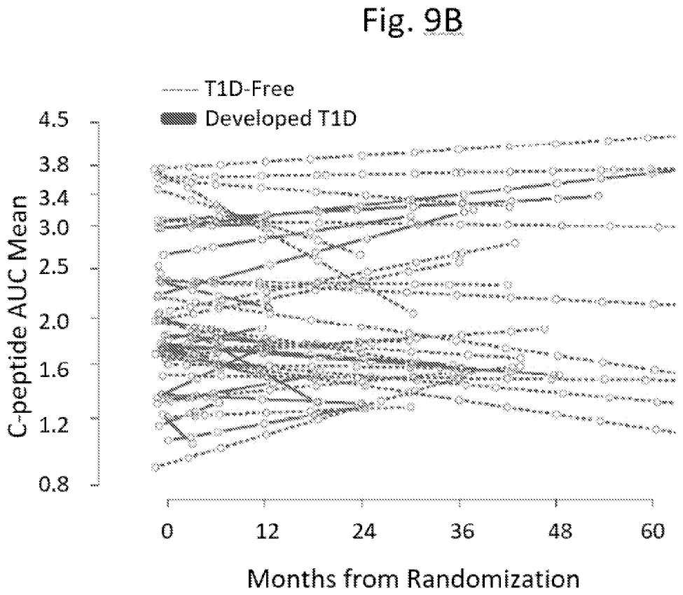

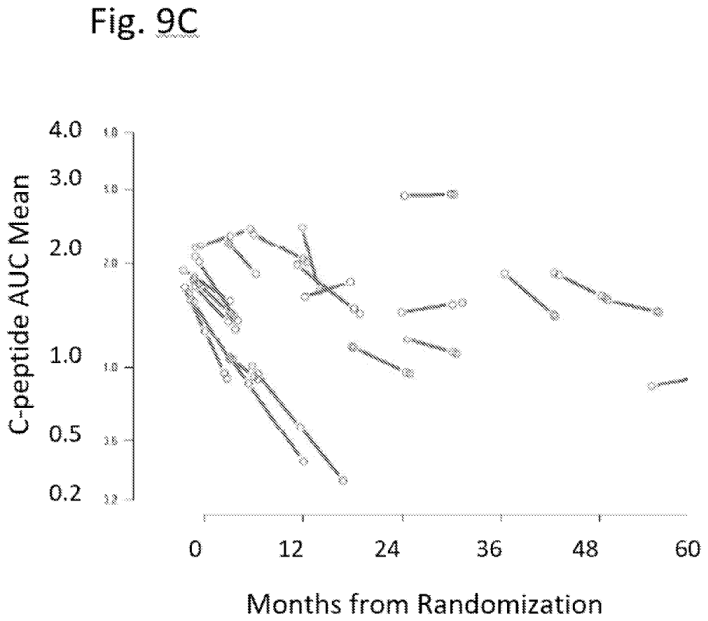

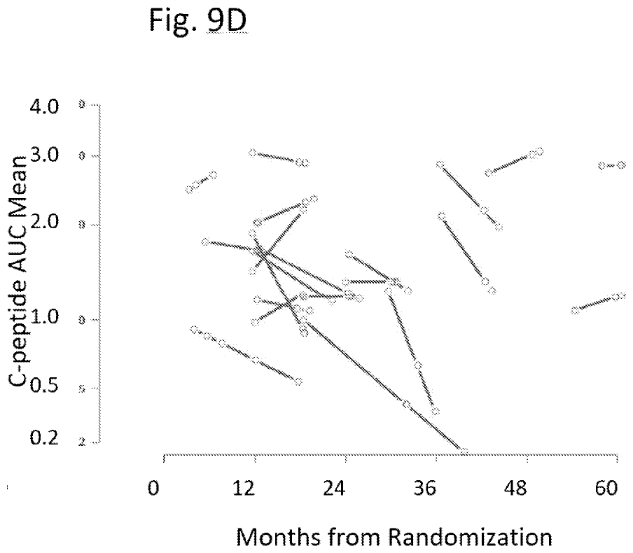

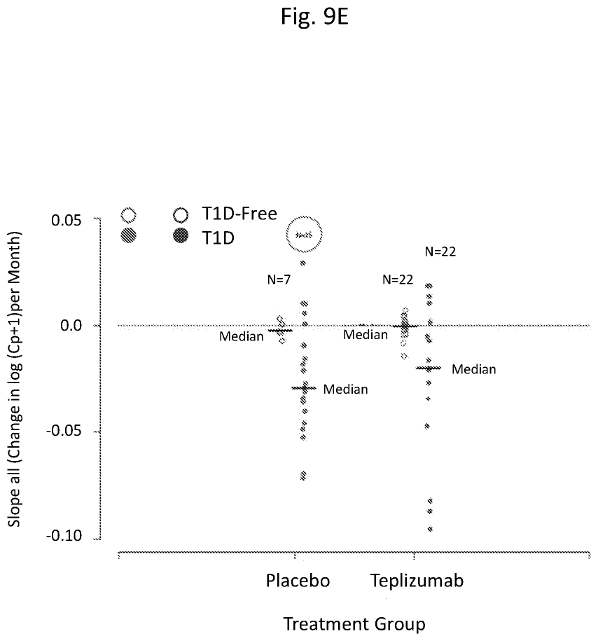

Preservation of C-peptide is maintained until the last 6 months preceding clinical diagnosis: To determine the duration of these metabolic effects, we analyzed the C-peptide trajectories (least-square lines) over the entire study period or until the 6 months prior to when the participant was diagnosed with T1D. (FIGS. 9A and 9B). With this analysis, C-peptide AUC continued to decline in the placebo group, with a median slope significantly less than 0 (median, IQR: −0.00382, −0.0107 to 0.000755, Wilcoxon 1-sample: p=0.04). The loss of C-peptide in the placebo group was even more pronounced in the 6 months between the penultimate and final OGTT (mean slope (IQR) of −0.0242 (−0.0469, −0.0041); significantly non-zero (Wilcoxon 1-sample: p=0.0001) (FIGS. 9C and 9E).

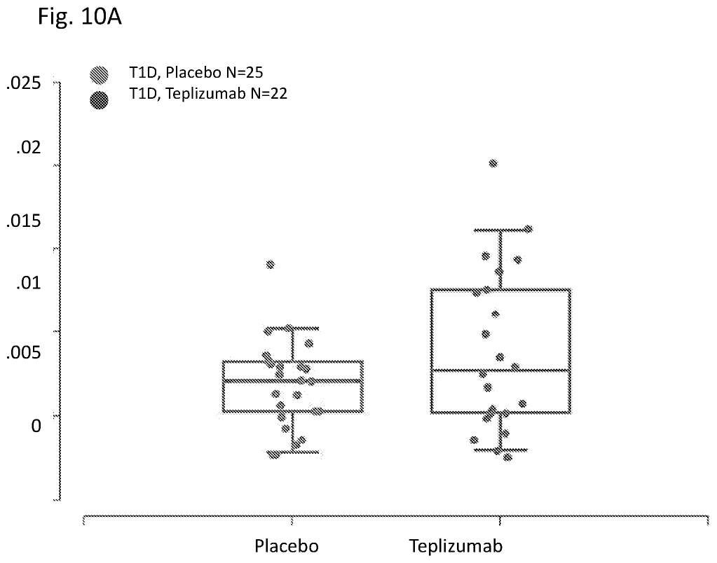

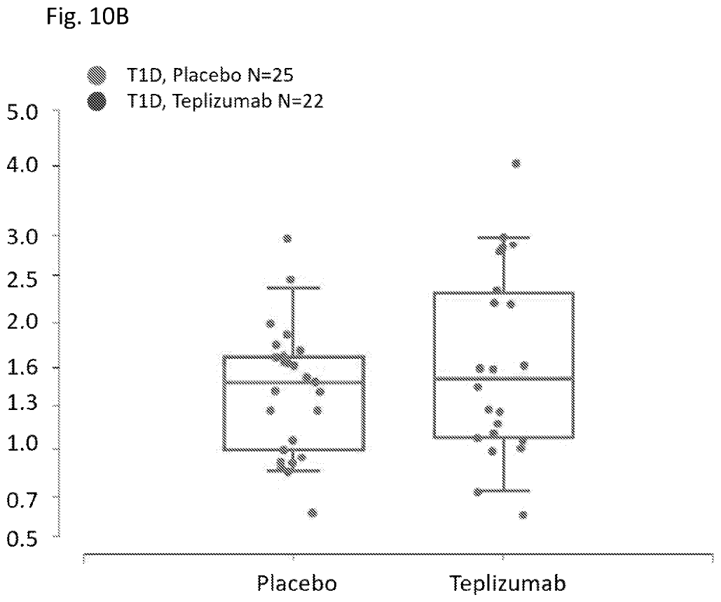

In contrast, the median slope for the teplizumab group until the end of the study period or until the 6 months prior to when the participant was diagnosed with T1D was not significantly different from 0 (mean (IQR): −0.000294 (−0.00372, 0.00304), Wilcoxon 1-sample: p=0.63) (FIG. 9B) and thus, less C-peptide AUC was lost over time compared to the placebo treated participants (Wilcoxon 2-sample: p=0.04). In the participants treated with teplizumab who were diagnosed with T1D, there was also a decline in the C-peptide AUC in the peridiagnostic period, but it was less pronounced than those in the placebo treatment arm who were diagnosed with T1D (mean slope (IQR): −0.0112, −0.0818, 0.0107), Wilcoxon 1-sample with a comparison to 0: p=0.09) (FIG. 9D) (Wilcoxon 2-sample comparing placebo and teplizumab slopes: p=0.06) (FIG. 9E). A difference in insulin sensitivity between the two treatment arms was not a likely explanation for these findings since the C-peptide AUC/glucose AUCs were similar in the teplizumab and placebo groups at the time of T1D diagnosis (p=0.23) (FIGS. 10A and 10B).

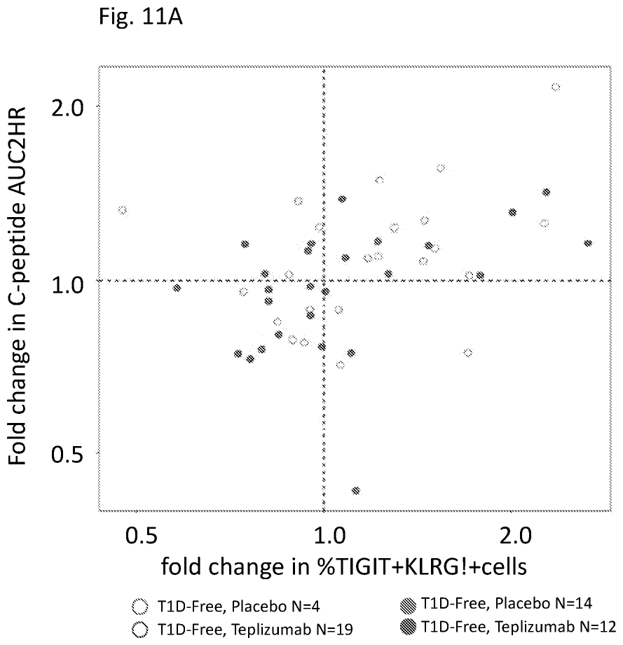

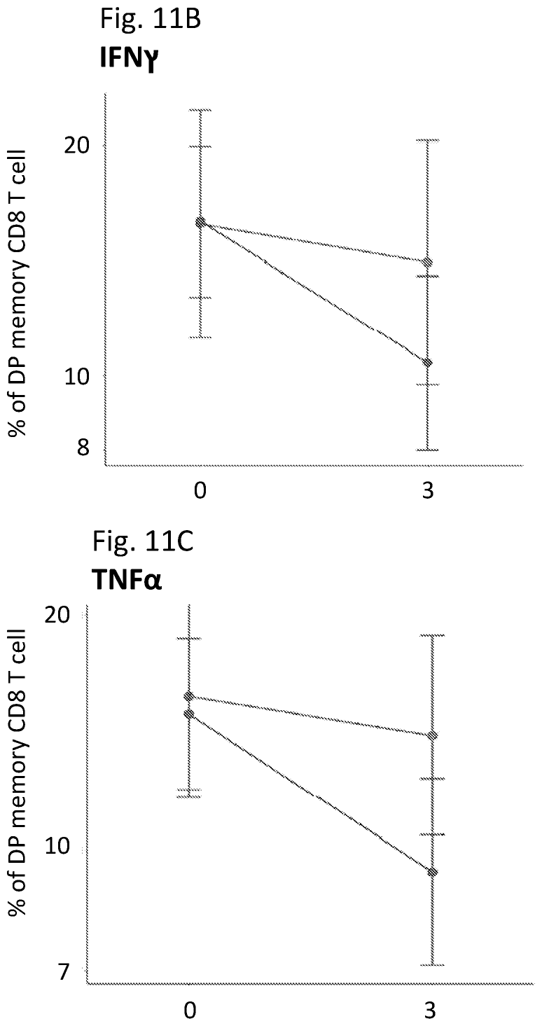

C-peptide responses correlate with increases in partially exhausted CD8+ T-cells: We postulated that the rapid improvement in metabolic responses was related to the effects of teplizumab on T-cells. We had previously described an increase in the frequency of memory CD8+ T-cells with teplizumab treatment that we proposed were “partially exhausted” by expression of TIGIT and KLRG1+ (double positive cells), and a transcriptional activation/exhaustion signature that could be further reduced by ligation of TIGIT (23, 25, 26, 37, 38). Thus, we checked if their frequency was associated with C-peptide AUC during or shortly after the drug treatment period and whether they were functionally exhausted. We observed a significant correlation of the change in frequency of CD8+KLRG1+TIGIT+ T-cells with the fold changes in C-peptide AUC at month 3, 6, and 18 (Table 6). The changes in T-cell subsets most likely preceded the changes in C-peptide and therefore, we also analyzed the fold changes in the double positive CD8+ T-cells at month 3 and the fold changes in C-peptide at month 6. There was a significant association between these two parameters in the drug but not placebo treated participants. (p=0.014) (FIG. 11A).

TABLE 6
Pearson Correlations Between % Change in
C-peptide and CD8+ T Cell Subpopulations

| | Month 3 | | Month 6 | | Month 18 | |
|---|---|---|---|---|---|---|
| CD8 Subsets | r | p | r | p | r | p |
| KLRG-1+TIGIT+ of | 0.429 | 0.016 | 0.433 | 0.01 | 0.463 | 0.011 |
| CD8 Central Memory | | | | | | |
| KLRG-1+TIGIT+ of | 0.421 | 0.018 | 0.460 | 0.006 | 0.461 | 0.012 |
| CD8 Effector Memory | | | | | | |

T cell exhaustion has been associated with reduced cytokine production following activation (39). We therefore measured intracellular cytokines after stimulation of PBMC with anti-CD3 and anti-CD28. Among the double positive CD8+ T-cells the frequency of IFNγ-producing (p<0.0001, p=0.0004) and TNFα-producing cells (p<0.0001 for both) were decreased at 3 (FIGS. 11B and 11C) and 6 months respectively in the teplizumab but not placebo treated participants. In contrast, relative proportions of IFNγ and TNFα producing cells among the double positive memory CD8+ T-cells remained stable in the placebo group at 3 and 6 months of follow-up. The fold decline in the frequency of the IFNγ and TNFα among the double positive CD8 memory cells between enrollment and month 3 were associated with the fold improvement in C-peptide between enrollment and month 6 (IFNγ: Pearson r=−0.29, p=0.164, TNFα: r=−0.39, p=0.056) (not shown).

### Discussion

Studies of natural history cohorts have described changes in metabolic function during the progression to T1D in relatives at risk. Our successful intervention trial, with teplizumab, in the at-risk population has given us a unique opportunity to directly assess how changing immune cells can affect metabolic function and progression to the clinical diagnosis of T1D in relatives at high-risk. In this extended follow up we show that the effects of the single 14-day course of teplizumab treatment persists: The median time to diabetes in the teplizumab group was approximately 5 years compared to approximately 2 years in the placebo treated participants, with 50% of the teplizumab treated participants vs 22% of placebo treated participants not diagnosed with T1D. Eighteen percent of the teplizumab-treated participants vs 6 percent of the placebo treated participants were followed for more than 5 years and were not diagnosed. Importantly, this is the first study to show successful modulation of the progression of beta cell failure prior to the diagnosis of T1D with immune intervention.

Although the participants enrolled in the trial had not been diagnosed with T1D, teplizumab treatment improved beta cell function. The average OGTT glucose levels were lower and C-peptide responses higher with teplizumab treatment. There was improvement in total and early insulin secretion rates, which identifies a functional as well as a quantitative improvement in insulin release. The early secretion of insulin, a feature of normal beta cell function, was the most dramatically changed, indicating that the impaired “beta cell glucose sensitivity” that has been described in patients who progress to clinical diabetes was improved (34). The metabolic changes were associated with an increased frequency of TIGIT+KLRG1+ memory CD8+ T-cells and a reduced secretion of cytokines (TNFα and IFNγ) that have been associated with pathology in T1D, indicating that the T-cells had functional exhaustion (23, 25, 26, 37, 38).

Because the clinical trial was designed as a time-to-event protocol, the variable time in the study for each participant created a challenge in analyzing the metabolic responses during the study OGTTs. Hence, we used the average on-study C-peptide, glucose, and HbA1c AUCs which included all of the available data for each participant.

Although the time in the trial was not a significant determinant of the average C-peptide AUC, there were time-dependent metabolic effects of the drug treatment. Beta cell function was declining in the participants as they were enrolling in the TN10 trial. Indeed, in previous studies we found that the level of beta cell death was high among similarly high-risk individuals and other studies have documented beta cell dysfunction in the peridiagnosis period (35, 40, 41). This metabolic data together with the relatively short median time to diagnosis of T1D in the placebo group indicates that the screening methods utilized identified an active time of disease and individuals at very high risk for progression. Consistent with preclinical studies, the effects at this period of active disease support the concept that this intervention may be most effective when there is immune cell activation (42). The greatest increase in C-peptide occurred shortly after teplizumab treatment, followed by stabilization of beta cell function, whereas in the placebo group, beta cell function declined gradually over time. Consistent with prior reports, in those who developed clinical diabetes in both treatment arms, there was a precipitous decline in the stimulated C-peptide levels seen about 6 months prior to T1D onset (40).

Unexpectedly, we did not find a relationship between the average on-study glucose AUC and C-peptide AUC. Moreover, the outcomes of the OGTTs fluctuated even within individuals that did and did not develop T1D. Most likely this variability reflects the tenuous level of residual insulin production. Consistent with this, the OGTTs did not uniformly normalize in those who were not diagnosed with T1D. Minor changes, such as in the kinetics of insulin secretion or host factors may change the outcomes of the OGTT which are categorically classified based on levels of glycemia associated with long-term microvascular complications and not necessarily beta cell function or insulin secretion (43). These clinical outcomes are similar to the effects of anti-CD3 mAb in the NOD model of T1D, prior to the diagnosis of diabetes, in which insulin granularity was improved but beta cell mass did not recover to normal levels (44, 45). Further studies with metabolic clamps might improve our analysis of the metabolic function, but such studies were impractical in this clinical trial setting. These findings also suggest that combining teplizumab with a drug that improves beta cell function with complementary mechanisms may be valuable. The factors that precipitated disease in the 6 months prior to clinical T1D diagnosis in both the treatment and placebo arms are not clear at this point. The similar relation between C-peptide and glucose in the two treatment arms among those who were diagnosed with T1D suggests that insulin insensitivity was not a precipitating factor for the diagnosis. Interestingly, even with progression to clinical diabetes, the decline in C-peptide tended to be less in the teplizumab vs placebo, which suggests that the effects of drug treatment on C-peptide may persist even during and potentially after the clinical diagnosis. There may be waning of the effects of the anti-CD3 antibody on immune cells which we identified previously by tracking the CD8 memory double positive cells (23). Other observations in the field show that progression to clinical diabetes is associated with acquisition of effector T-cell function, but it is possible that in this setting restored effector function may involve waning of the immune effects of teplizumab or even new or regenerated pathologic T-cells repopulating the repertoire after the single course of drug. The median age at the time of treatment in the TN10 study was 13.9 years, and in young children, the thymic output of T-cells may be ongoing. In other studies of the long term outcomes of patients treated with teplizumab, there was an increase frequency of Programmed Cell Death Protein 1 (PD-1)+ memory CD8+ T-cells in responders compared to non-responders and controls suggesting that changes in the phenotype and function of the CD8+ memory compartment may occur over time (46). Ongoing work tracking TCRs and single cell analyses will help to address these hypotheses and may suggest agents that could be used to extend the diabetes-free period possibly by blocking pathways needed for T-effector expansion (47).

There are limitations to our studies. The number of subjects was relatively small, and the study was powered to detect differences in diabetes incidence rather than changes in C-peptide AUC, insulin secretion, and immune function. Additionally, the time-to-event design of the original study had some important implications for the analyses included here. We did not have OGTT analyses for most individuals after diagnosis of T1D which limited our ability to compare OGTT data between all members of placebo and teplizumab groups over the same time period, particularly for the placebo group, which exhibited more rapid progression to diabetes. The time-to-event design also limited our ability to compare the relationship between metabolic endpoints and T1D progression, as some individuals included in the study that did not progress to diabetes may ultimately develop T1D. Furthermore, given prior results showing teplizumab treatment preserved C-peptide in patients with recent-onset T1D (19-25), positive effects on C-peptide might also be expected to occur amongst individuals that developed diabetes during this study. Participants from both arms of the trial were enrolled in the TrialNet LIFT study, which performs longitudinal metabolic testing in participants who have been diagnosed with T1D (13). In summary, we show extended delay in progression to T1D in at-risk subjects treated with teplizumab. Teplizumab treatment changed the biologic course of the disease by enhancing beta cell function reflected by the quantitative and qualitative improvements in insulin secretion. These changes were associated with modulation of the frequency and function of memory CD8+ T-cells. The pronounced early efficacy of the drug followed by stabilization of beta cell function also suggests that repeated treatment with teplizumab or the addition of other complimentary agents at key timepoints in the clinical course may be valuable to extend the delay or even prevent the diagnosis of T1D. Finally, our findings have implications for other autoimmune diseases by showing how immune intervention can change the pathobiology even prior to disease diagnosis and lead to a clinically significant outcome.

### Materials and Methods

### Trial Design

The design of this phase 2, randomized, placebo-controlled, double-blind trial (NCT01030861) has previously been reported (26). Institutional-review-board approval was obtained at each participating site. The participants, their parents, or both provided written informed consent or assent before trial entry. The participants were identified through the TrialNet Pathway to Prevention study (TN01) (14, 48). In that study, OGTTs were performed at approximately 6-month intervals in islet autoantibody positive individuals (including anti-glutamic acid decarboxylase 65, micro insulin, anti-islet antigen 2, anti-zinc transporter 8, and/or islet-cell antibodies), and the glucose results from these tests were used to identify eligibility for the anti-CD3 prevention trial (TN10) and were used in this data analysis. Islet autoantibody testing, HLA genotyping, and OGTT testing were performed as previously described (4, 49).

Briefly, eligibility criteria included age >=8 years at randomization, history of a relative with type 1 diabetes, positive titers for two or more islet autoantibodies, and dysglycemia on OGTT (fasting glucose 110-125 mg/dL (6.1-6.9 mmol/L), a 2-hour postprandial plasma glucose level of >=140 mg/dL (7.8 mmol/L) and <200 mg/dL (11.1 mmol/L), or an intervening postprandial glucose level at 30, 60, or 90 minutes of >200 mg/dL. For participants who did not have a hemoglobin A1c available at the baseline visit, values obtained within the 3 months before treatment were utilized.

Participants were randomly assigned to teplizumab or saline and treated with a 14-day outpatient course administered as an IV infusion in a clinical research center. Teplizumab was dosed at 51 ug/m² on day 0, 103 ug/m² on day 1, 207 ug/m² on day 2, 413 ug/m² on day 3, followed by a dose of 826 ug/m² on days 4 through 13. OGTTs were performed 3 months and 6 months after the infusions and every 6 months thereafter. Random screening glucose levels were evaluated at 3-month intervals, and an OGTT was performed if the random glucose level was >200 mg/dL (11.1 mmol/). T1D was diagnosed using ADA criteria during an OGTT but only after the diabetic OGTT was sequentially confirmed. The date of diagnosis was identified as the time of the first of the 2 diagnostic tests (50). Six participants were clinically diagnosed with T1D outside of OGTT monitoring. The original trial end date was May 2019. Participants who had not been diagnosed with T1D were transferred into the TrialNet Pathway to Prevention Natural History study (TN01) for follow up OGTT monitoring. Data from that follow up, between July 2011 and March 2020 are included in this analysis. Participants that did develop T1D were offered enrollment in TrialNet's Long Term Investigational Follow-up (LIFT) study for continued metabolic follow-up.

### Metabolic Analyses

OGTT C-peptide and glucose values were tested by Northwest Lipids Research Laboratories using the TOSOH and Roche C-peptide and glucose immunoassays, respectively. OGTT results were assigned to the nearest study visit timepoint (within 3 months of the official timepoint assignment. OGTT results were classified as normal, dysglycemic, or diabetic based on above definitions used for study entry. The baseline OGTT was the study at the time or immediately prior to randomization.

Area under the curve (AUC) values for ISR, C-peptide, and glucose were calculated using the trapezoidal rule. The on-study AUC means for C-peptide, glucose, and HbA1c were calculated by multiplying the AUC means for each OGTT visit and the visit intervals in days (as the trapezoidal base) to calculate a total study AUC, and then dividing by the days from the first to the last OGTT (confirmatory diabetic OGTT if developed T1D). Insulin secretory rates (ISRs) were calculated using the Chronobiological Series Analyzer (CSA) software, which uses a 2-compartment model for hormone clearance and standard kinetic parameters for C-peptide (51-53). ISR calculations were performed using participant OGTT C-peptide and glucose values, as well as age, sex, height, and weight. The insulin secretion was divided into the amount (pmol) secreted over the 2-hour OGTT or in the first or second hour of the test.

### Flow Cytometry Analysis

Peripheral blood mononuclear cells (PBMC) were processed and stored at the NIDDK repository. Cryopreserved vials of PBMC were sent to ITN Core laboratory at Benaroya Research Institute for analysis by flow cytometry with antibody panels shown in Tables 7 and 8. T-cell phenotyping was performed on thawed PBMC and the frequency of CD45RO+CD8+ T-cells that were TIGIT+KLRG1+CD57− was determined as described previously (54). Intracellular cytokine expression was measured after 6 hours with stimulation of PBMC by plate-bound anti-CD3 (1 μg/ml) and soluble anti-CD28 (10 μg/ml) in the presence of equimolar amounts of Golgi-stop. The frequency of TIGIT+KLRG1+CD8+ memory (CD45RA−) T-cells that produce IFNγ or TNFα were determined at baseline and month 3.

TABLE 7
T cell phenotype flow cytometry panel

| Markers | Format | Clone | Vendor* |
|---|---|---|---|
| CD56 | BUV395 | NCAM16.2 | BD |
| CD45RA | BUV737 | HI100 | BD |
| Ki67 | BV421 | Ki-67 | BL |
| CCR7 | BV510 | G0434H7 | BL |
| CD3 | BV605 | OKT3 | BL |
| PD1 | BV650 | EH12.2H7 | BL |
| CD127 | BV711 | A019D5 | BL |
| CD-45R0 | BV786 | UCHL1 | BD |
| CD4 | BB515 | RPA-T4 | BD |
| Eomes | PE | WD1928 | eBio |
| FoxP3 | PE-CF594 | 259D/C7 | BD |
| KLRG1 | PE-Vio770 | REA261 | Miltenyi |
| TIGIT | APC | MBSA43 | eBio |
| CD8 | Ax700 | SKI | BL |
| CDS7 | APC-Vio770 | REA769 | Miltenyi |
| L/D | BUV496 | | |

*BL = Biolegend, BD = Becton Dickinson, Miltenyi = Miltenyi biotech, eBio = eBioscience

TABLE 8
Intracellular cytokine staining flow cytometry panel.

| Markers | Format | Clone | Vendor* |
|---|---|---|---|
| IL-17 | BV421 | BL168 | BL |
| CD45RA | BV605 | HI100 | BL |
| GrzmB | BV510 | GB11 | BD |
| PD-1 | BV650 | EH12.2H7 | BL |
| CD3 | BUV737 | UCHT1 | BD |
| TNFa | APC | MAb11 | BL |
| CD127 | BV785 | A019D5 | BL |
| CD4 | Ax 700 | RPA-T4 | BL |
| IFNg | BV711 | B27 | BD |
| FoxP3 | PE-CF594 | 259D/C7 | BD |
| KLRG1 | FITC | SA231A2 | BL |
| TIGIT | PE-Cy7 | A15153G | BL |
| CD8 | BUV395 | RPA-T8 | BD |
| IL-2 | BB700 | MQ1-17H12 | BD |
| L/D | BUV496 | | |

*BL = Biolegend, BD = Becton Dickinson, Miltenyi = Miltenyi biotech, eBio = eBioscience

Instrument standardization was performed using 8 peak rainbow calibration beads (Spherotech, Lake Forest, IL) adjusting PMT voltages for consistent 7^{th} peak mean fluorescent intensities. All samples from the same subject were run on the same day, and an internal control arm from the same subject was run each week. Sample acquisition was performed as previously described on an LSR-Fortessa (BD Biosciences) with FACS Diva software and analyzed with FlowJo software version 9.5 (Tree Star, Ashland, OR) (54). The quadrants were placed based on staining controls. Gated populations with <100 events were excluded from analysis.

### Statistical Analysis

The original trial was designed as a time-to-event analysis and therefore participants who were diagnosed with T1D were not followed further in that study. The impact of teplizumab treatment on incidence of type 1 diabetes following enrollment was performed using a Cox proportional hazards model. For this analysis, metabolic parameters over the entire period of the trial included OGTT data in the visit immediately prior to and all OGTT data after study drug treatment (confirmatory diabetic OGTT for individuals diagnosed with diabetes, or last available OGTT for those remaining diabetes free). Slopes for changes in glucose and C-peptide prior to and after enrollment were calculated using linear regression analysis of available OGTT visit data for specified intervals. An impact of treatment on each endpoint was determined by fitting results to an ANCOVA model, with age, baseline value, and treatment group included as covariates. Wald tests were used to determine if covariates significantly impacted the model.

Estimated slopes for changes in the insulin secretion rates were also calculated for each subject based on changes before treatment (time points up to 6 months prior to baseline) and for after initiation of treatment (time points up to 6 months after baseline) using linear regression models as well as mixed models for repeated measures. Insulin secretion rates were calculated across the overall 2-hour interval as well as specifically for the first hour and the second hour intervals of the OGTTs. Differences in these slopes before vs. after treatment were compared using Wilcoxon signed rank tests within and across treatment arms. Differences and percent changes in these slopes before vs. after treatment were also evaluated using a generalized linear model to assess the influence of treatment arm.

Flow cytometry data were log-transformed for statistical analysis. Pearson's correlation coefficient was calculated to determine associations between fold changes in C-peptide AUC and frequency of TIGIT+KLRG1+ CD8+ memory T-cells. The frequency of TIGIT+KLRG1+ CD8+memory T-cells producing IFNγ or TNFα were analyzed by paired t-test.

### Example 2: Anti-CD3 Antibody (Teplizumab), Delays Type 1 Diabetes Onset in Stage 2 Type 1 Diabetes

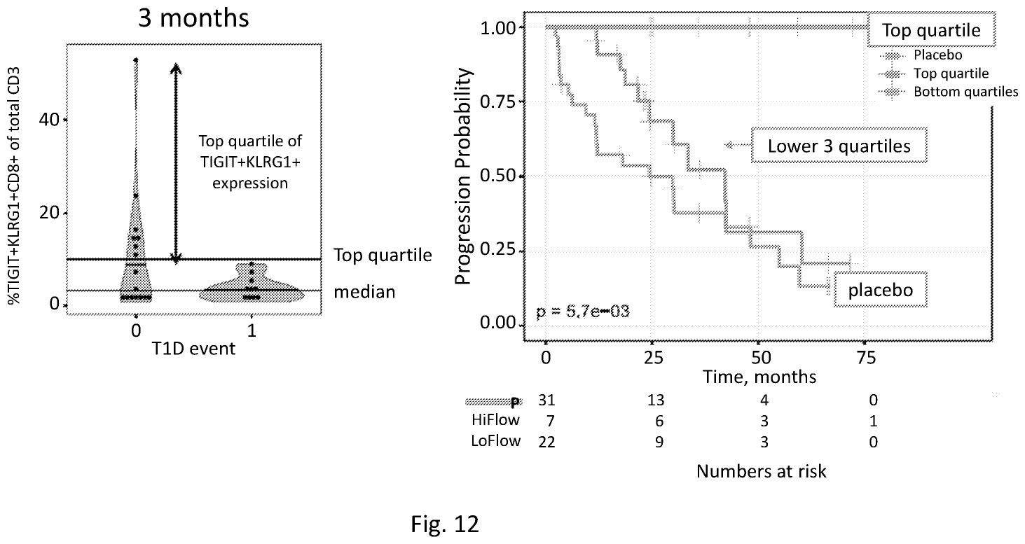

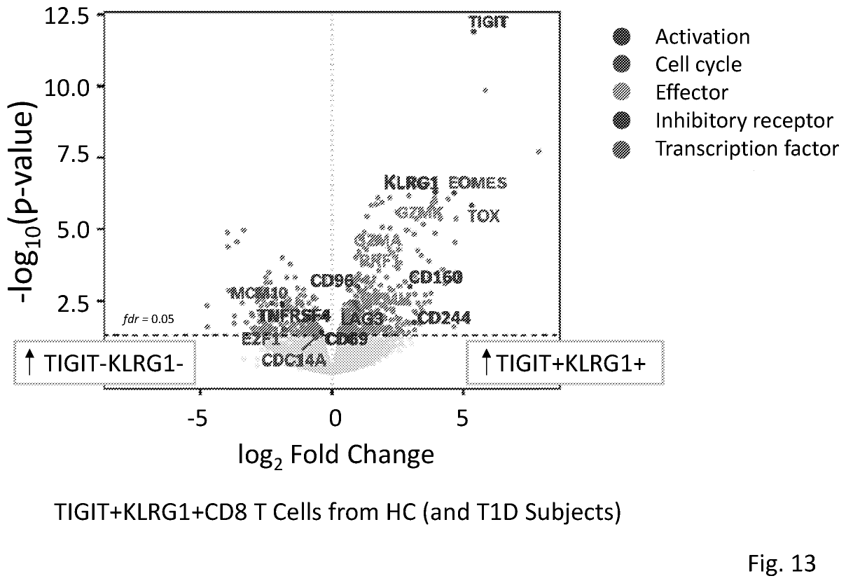

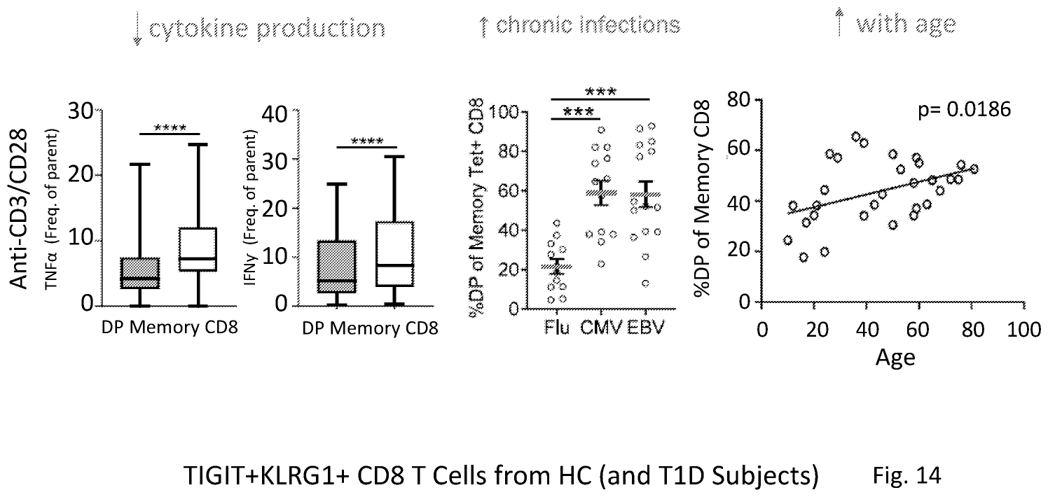

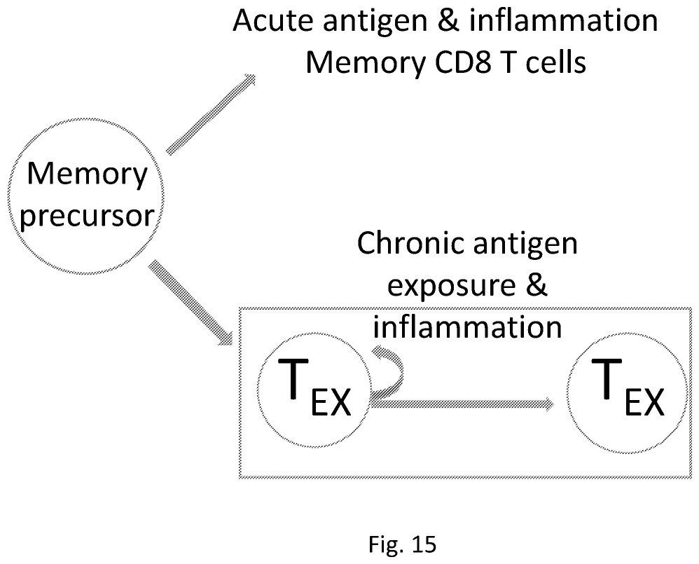

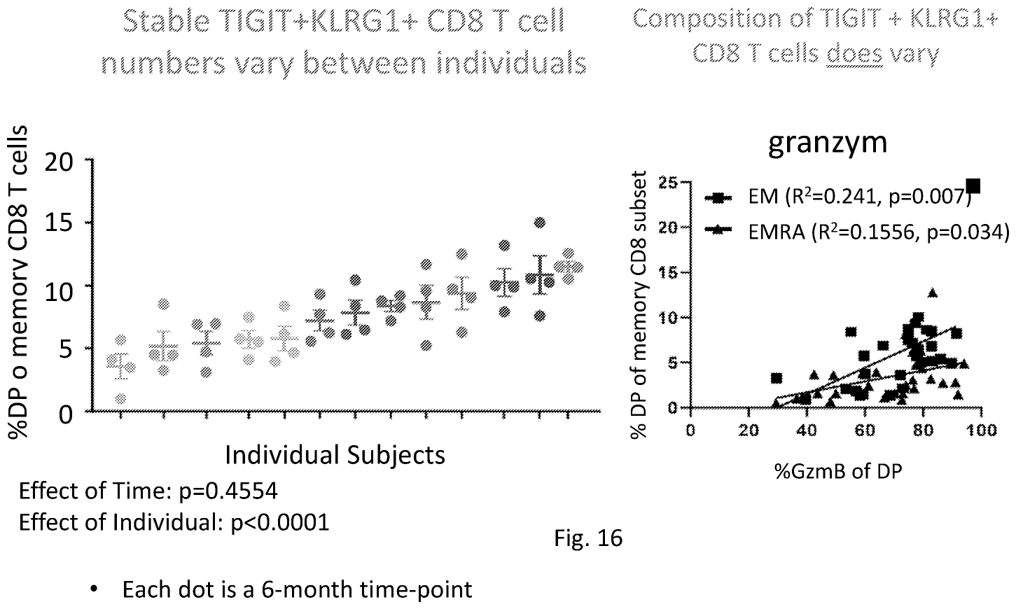

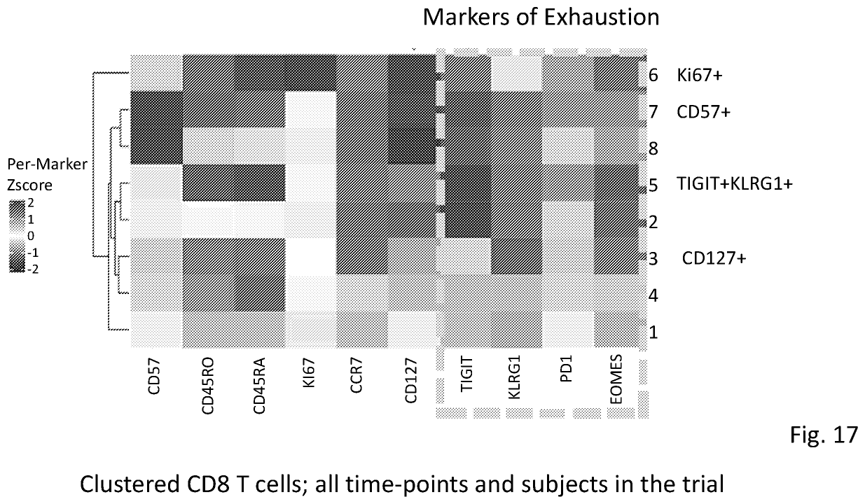

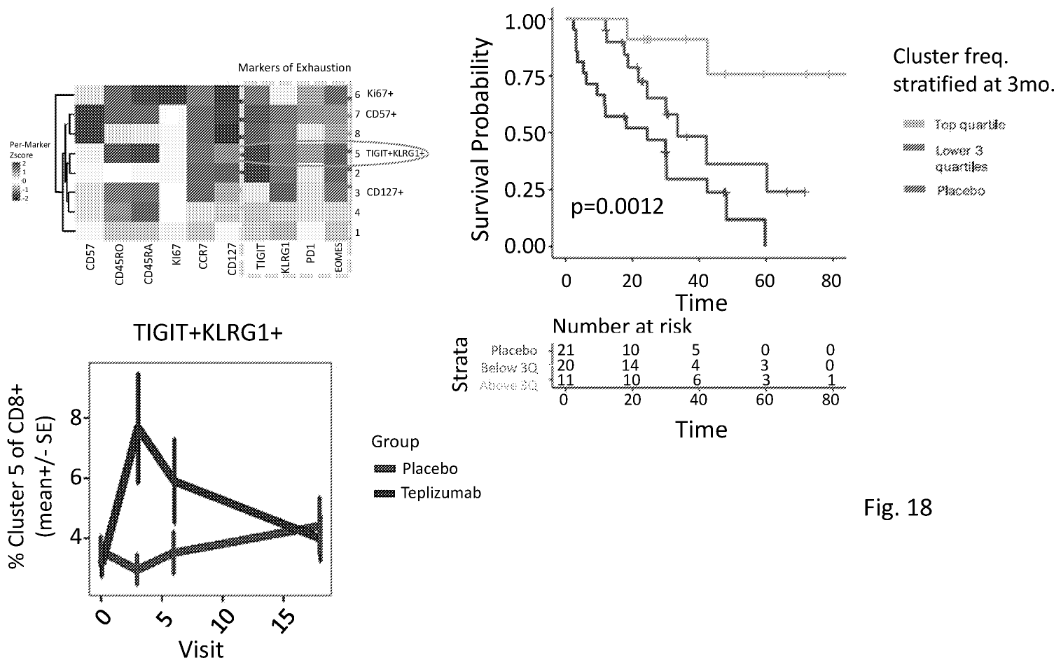

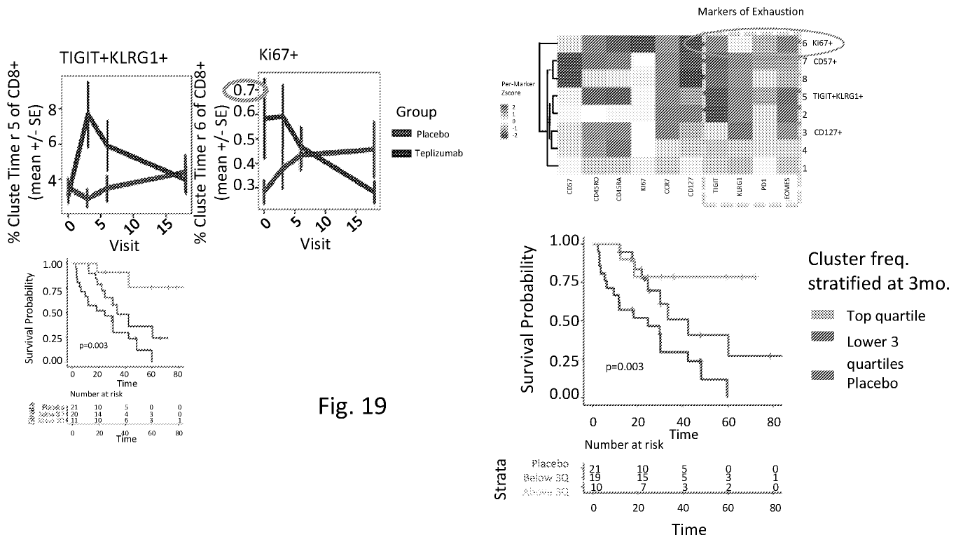

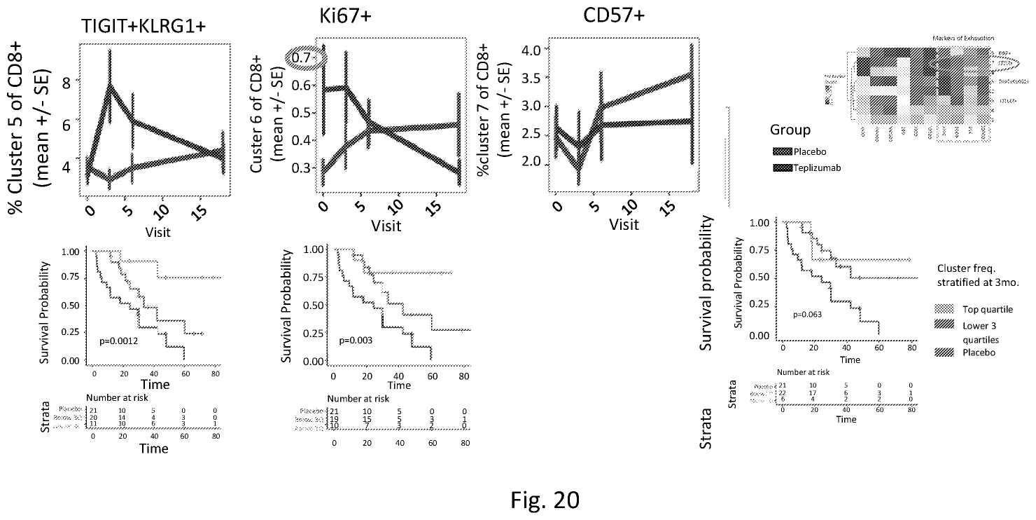

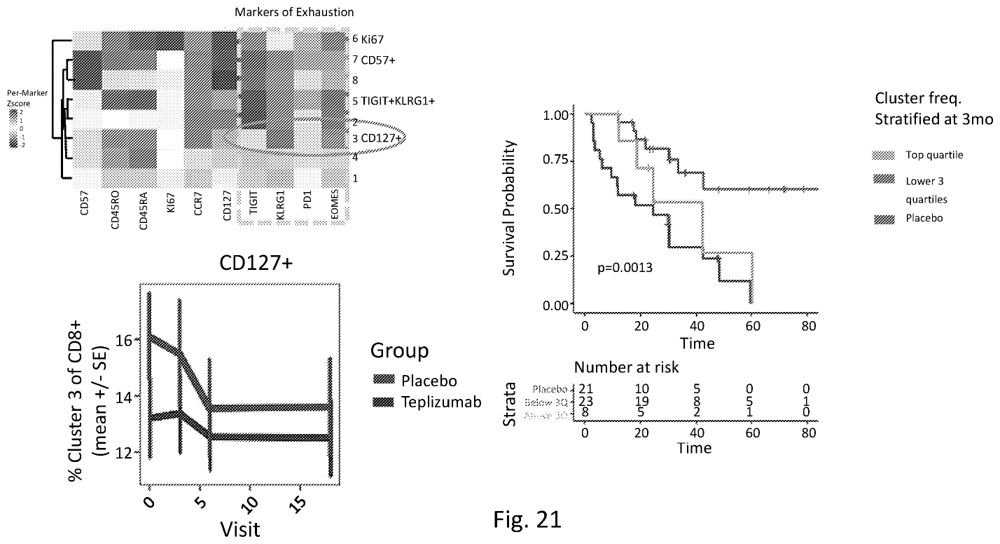

As shown in FIGS. 12-21, in at-risk (Stage 2) relatives of T1D patients, a single course of teplizumab (as described in Example 1) increased TIGIT+ KLRG1+ exhausted CD8+T-cells correlating with delay/prevention of clinical T1D. Surprisingly, no conversion to clinical T1D was observed in subjects with >10% exhausted CD8+ T-cells (% in all CD3+ T-cells, i.e., top quartile of exhausted T-cells) in the circulation 3 months post teplizumab treatment (p=0.005). Subjects with more TIGIT+KLRG1+ CD8+ T-cells 3 months following treatment with teplizumab responded the best.

These cells may express other markers of exhaustion such as PD1 and Eomes. These cells may be partly exhausted, and produce lower levels of inflammatory cytokines than in placebo-treated patients.

TIGIT+KLRG1+ CD8 T-Cells are not homogeneous, but instead, vary across individuals in number and function. Rather than being uniformly exhausted, TIGIT+KLRG1+ cells are a mixture of less-and more-functional populations.

We also observed a decrease in CD8+ T-cells expressing the proliferation markers Ki67 and CD57 compared to placebo, consistent with the reduction of effector T-cells and increase in exhausted T-cells. The decrease in Ki67 and CD57 correlates with clinical response (p=0.003 and p=0.006 respectively). CD127+ CD8 T-cells are associated with worse outcome.

Thus, in some embodiments, teplizumab can be dosed repeatedly and used in combinations to increase the generation/maintenance of exhausted T-cells and improve response and outcomes.

In some embodiments, teplizumab responsiveness can be predicted before dosing or shortly after dosing by determination of exhausted T-cells.

### Example 3: 2D Analysis of Glucose and C-Peptide Shows a Teplizumab Effect in Individuals at Risk for T1D 3 Months After Treatment

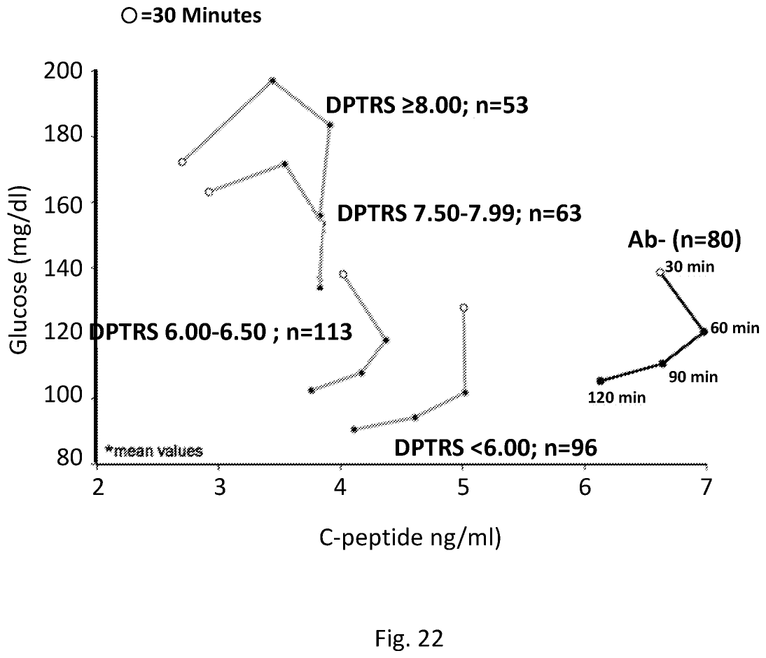

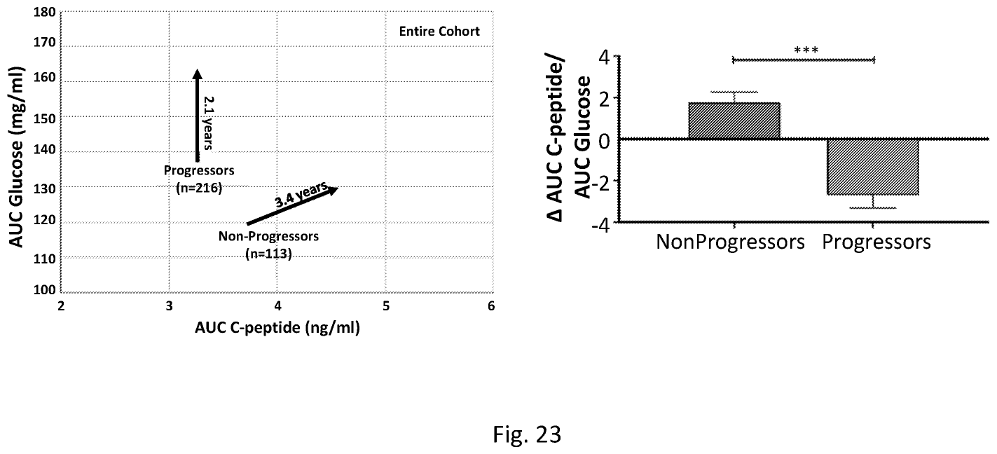

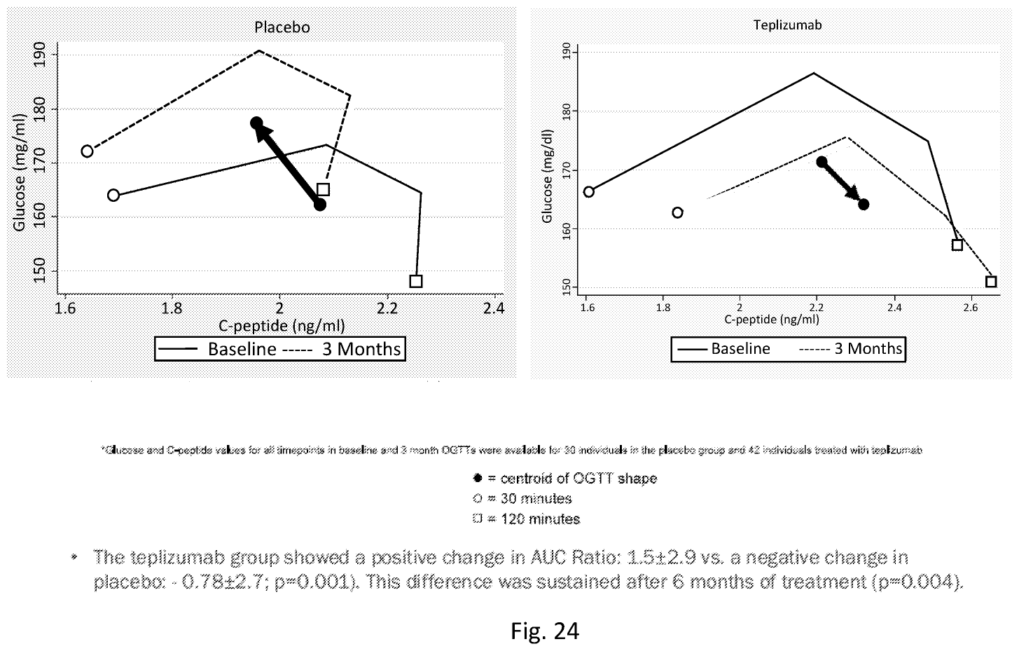

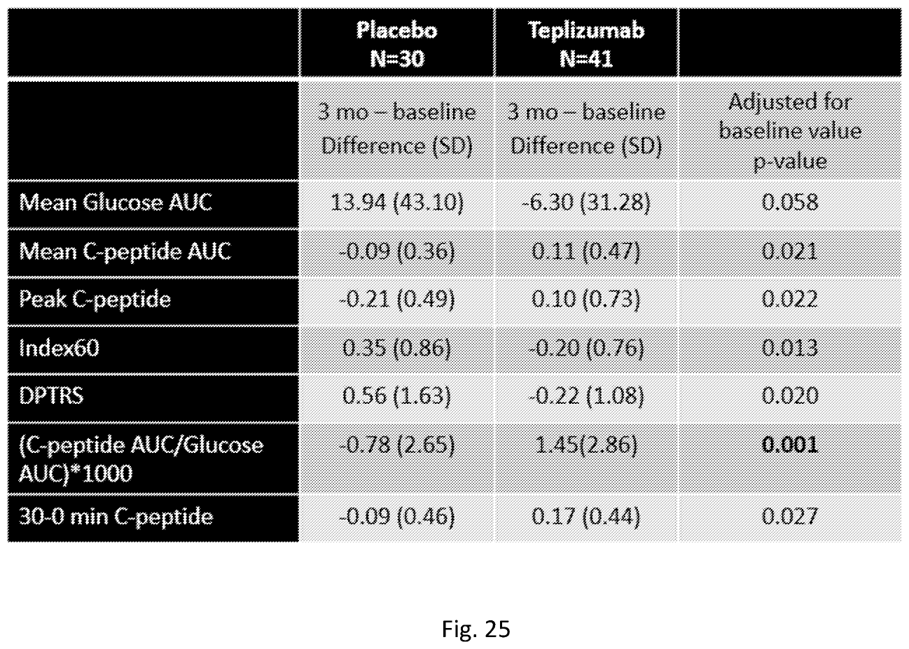

As shown in FIGS. 22-25, in at-risk (Stage 2) relatives of T1D patients, a single course of teplizumab (as described in Example 1) increased C-peptide AUC/glucose AUC ratio for at least 6 months. At 3 months post-dosing, increase in teplizumab arm (1.5±2.9) vs. decrease in placebo (0.78±2.7; p=0.001). This difference was sustained 6 months post-treatment (p=0.004). Thus, this ratio can be used as a new early endpoint (e.g., at 3 months) for future studies of teplizumab (monotherapy re-treatment, combinations).

Teplizumab also decreased the Diabetes Prevention Trial Risk Score (DPTRS) (see Sosenko et al., Diabetes Care (2012) 35(7):1552-5, incorporated herein by reference). This is consistent with benefit on C-peptide and glycemia: +0.56 on placebo and −0.22 in teplizumab (p=0.02). The DPTRS can also be used to help guide re-dosing in at risk individuals.

### Example 4: Clinical Pharmacokinetics and Pharmacodynamics

Mechanism of Action: Teplizumab is a humanized monoclonal antibody that targets the cluster of differentiation 3 (CD3) antigen, which is co-expressed with the T-cell receptor (TCR) on the surface of T lymphocytes. Though the mechanism of action of teplizumab for the proposed indication has not been confirmed, it appears to involve weak agonistic activity on signaling via the TCR-CD3 complex, which is thought to expand regulatory T-cells and re-establish immune tolerance.

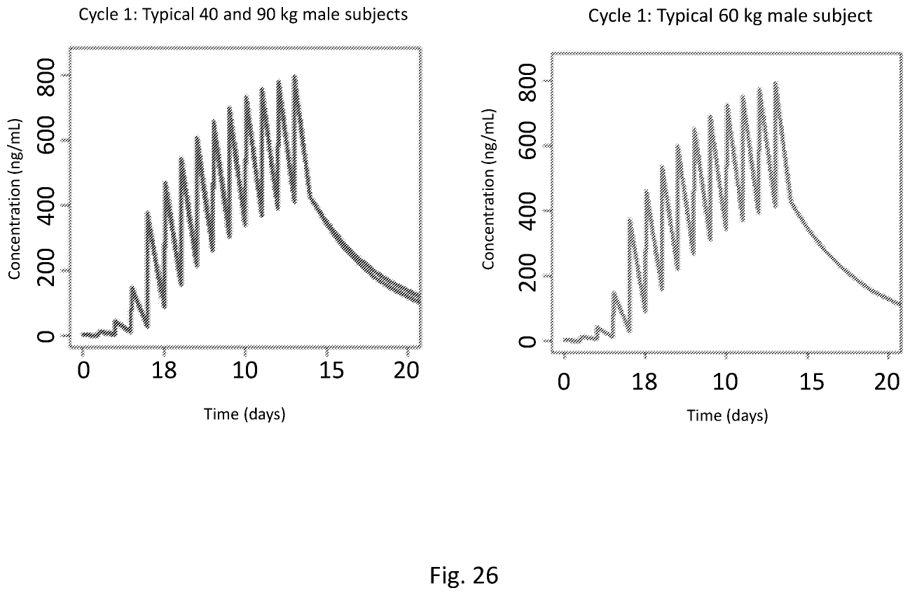

Pharmacokinetics: FIG. 26 shows plots of predicted mean teplizumab concentrations over time using a 14-day intravenous (IV) dosing regimen with a 4-day ramp-up followed by repeated doses of 826 μg/m² on Days 5 to 14. The left panel represents a typical 60 kg male subject and the right panel represents a typical 40 kg and 90 kg male subject. Body surface area (BSA)-based dosing normalizes the exposure across body size.

The repeated IV infusions resulted in increasing serum teplizumab levels, although steady-state PK was not achieved at the end of dosing (Day 14 with this dosing regimen). The average accumulation ratio for area under the curve (AUC) between Day 5 and Day 14 was 3.4. The predicted mean (±SD) total AUC for the 14-day dosing regimen was 6421±1940 ng·day/mL with C max and C min of 826±391 and 418±225 ng/ml, respectively, on Day 14.

Distribution: The central and peripheral volume of distribution from population PK analysis was 3.4 L and 6.9 L, respectively.

Elimination: Teplizumab clearance is not dose-proportional, likely driven by its saturable binding to CD3 receptors on the T-cell surface. Teplizumab is expected to be degraded into smaller peptide fragments by catabolic pathways. The clearance of teplizumab following the 14-day dosing regimen was estimated from population PK analysis to be 2.3 L/day, with a terminal half-life of approximately 4 days.

The planned commercial drug product is manufactured in a different facility from the clinical trial product and was not used in the clinical studies submitted to support efficacy and safety. A single-dose PK bridging study was conducted in healthy volunteers that evaluated the biocomparability of the commercial drug product with the clinical trial drug product. The mean AUC0-inf for the commercial product was less than half (48.5%, 90% CI: 43.6 to 54.1) of the AUC0-inf for the product used in the primary efficacy study. The reason for this difference seems to be faster clearance of the drug from circulation rather than differences in the strength of the product, as similar concentrations were observed immediately following IV infusion (C max of the commercial product was 94.5% (90% CI: 84.5 to 106) of that observed in the clinical trial drug product).

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

### REFERENCES

1. A. G. Ziegler, M. Rewers, O. Simell, T. Simell, J. Lempainen, A. Steck, C. Winkler, J. Ilonen, R. Veijola, M. Knip, Seroconversion to multiple islet autoantibodies and risk of progression to diabetes in children. Jama 309, 2473-2479 (2013).
2. R. A. Insel, J. L. Dunne, M. A. Atkinson, J. L. Chiang, D. Dabelca, P. A. Gottlieb, C. J. Greenbaum, K. C. Herold, J. P. Krischer, A. Lernmark, R. E. Ratner, M. J. Rewers, D. A. Schatz, J. S. Skyler, J. M. Sosenko, A. G. Ziegler, Staging presymptomatic type 1 diabetes: a scientific statement of JDRF, the Endocrine Society, and the American Diabetes Association. Diabetes Care 38, 1964-1974 (2015).
3. J. P. Krischer, K. F. Lynch, A. Lernmark, W. A. Hagopian, M. J. Rewers, J. X. She, J. Toppari, A. G. Ziegler, B. Akolkar, T. S. Group, Genetic and Environmental Interactions Modify the Risk of Diabetes-Related Autoimmunity by 6 Years of Age: The TEDDY Study. Diabetes Care 40, 1194-1202 (2017).
4. J. L. Mahon, J. M. Sosenko, L. Rafkin-Mervis, H. Krause-Steinrauf, J. M. Lachin, C. Thompson, P. J. Bingley, E. Bonifacio, J. P. Palmer, G. S. Eisenbarth, J. Wolfsdorf, J. S. Skyler, C. TrialNet Natural History, G. Type 1 Diabetes TrialNet Study, The TrialNet Natural History Study of the Development of Type 1 Diabetes: objectives, design, and initial results. Pediatr Diabetes 10, 97-104 (2009).
5. H. T. Siljander, R. Hermann, A. Hekkala, J. Lahde, L. Tanner, P. Keskinen, J. Ilonen, O. Simell, R. Veijola, M. Knip, Insulin secretion and sensitivity in the prediction of type 1 diabetes in children with advanced beta-cell autoimmunity. Eur J Endocrinol 169, 479-503 (2013).
6. N. C. Foster, R. W. Beck, K. M. Miller, M. A. Clements, M. R. Rickels, L. A. DiMeglio, D. M. Maahs, W. V. Tamborlane, R. Bergenstal, E. Smith, B. A. Olson, S. K. Garg, State of Type 1 Diabetes Management and Outcomes from the T1D Exchange in 2016-2018. Diabetes Technol Ther 21, 66-72 (2019).
7. A. Rawshani, N. Sattar, S. Franzen, A. Rawshani, A. T. Hattersley, A. M. Svensson, B. Eliasson, S. Gudbjornsdottir, Excess mortality and cardiovascular disease in young adults with type 1 diabetes in relation to age at onset: a nationwide, register-based cohort study. Lancet 392, 477-486 (2018).
8. S. J. Livingstone, D. Levin, H. C. Looker, R. S. Lindsay, S. H. Wild, N. Joss, G. Leese, P. Leslie, R. J. McCrimmon, W. Metcalfe, J. A. McKnight, A. D. Morris, D. W. Pearson, J. R. Petric, S. Philip, N. A. Sattar, J. P. Traynor, H. M. Colhoun, g. Scottish Diabetes Research Network epidemiology, R. Scottish Renal, Estimated life expectancy in a Scottish cohort with type 1 diabetes, 2008-2010. JAMA 313, 37-44 (2015).
9. B. Tao, M. Pietropaolo, M. Atkinson, D. Schatz, D. Taylor, Estimating the cost of type 1 diabetes in the U.S.: a propensity score matching method. PLOS One 5, e11501 (2010).
10. C. Evans-Molina, E. K. Sims, L. A. DiMeglio, H. M. Ismail, A. K. Steck, J. P. Palmer, J. P. Krischer, S. Geyer, P. Xu, J. M. Sosenko, G. Type 1 Diabetes TrialNet Study, beta Cell dysfunction exists more than 5 years before type 1 diabetes diagnosis. JCI Insight 3, (2018).
11. E. K. Sims, L. A. DiMeglio, Cause or effect? A review of clinical data demonstrating beta cell dysfunction prior to the clinical onset of type 1 diabetes. Mol Metab 27S, S129-S138 (2019).
12. M. K. Koskinen, O. Helminen, J. Matomäki, S. Aspholm, J. Mykkänen, M. Mäkinen, V. Simell, M. Vähä-Mäkilä, T. Simell, J. Ilonen, Reduced β-cell function in early preclinical type 1 diabetes. European Journal of Endocrinology 174, 251-259 (2016).
13. P. J. Bingley, D. K. Wherrett, A. Shultz, L. E. Rafkin, M. A. Atkinson, C. J. Greenbaum, Type 1 Diabetes TrialNet: A Multifaceted Approach to Bringing Disease-Modifying Therapy to Clinical Use in Type 1 Diabetes. Diabetes Care 41, 653-661 (2018).
14. C. J. Greenbaum, C. Speake, J. Krischer, J. Buckner, P. A. Gottlieb, D. A. Schatz, K. C. Herold, M. A. Atkinson, Strength in Numbers: Opportunities for Enhancing the Development of Effective Treatments for Type 1 Diabetes-The TrialNet Experience. Diabetes 67, 1216-1225 (2018).
15. B. M. Nathan, D. Boulware, S. Geyer, M. A. Atkinson, P. Colman, R. Goland, W. Russell, J. M. Wentworth, D. M. Wilson, C. Evans-Molina, D. Wherrett, J. S. Skyler, A. Moran, J. M. Sosenko, T. Type 1 Diabetes, G. Diabetes Prevention Trial-Type 1 Study, Dysglycemia and Index60 as Prediagnostic End Points for Type 1 Diabetes Prevention Trials. Diabetes Care 40, 1494-1499 (2017).
16. J. M. Sosenko, J. P. Palmer, L. Rafkin-Mervis, J. P. Krischer, D. Cuthbertson, J. Mahon, C. J. Greenbaum, C. C. Cowie, J. S. Skyler, G. Diabetes Prevention Trial-Type 1 Study, Incident dysglycemia and progression to type 1 diabetes among participants in the Diabetes Prevention Trial-Type 1. Diabetes Care 32, 1603-1607 (2009).
17. J. P. Palmer, C-peptide in the natural history of type 1 diabetes. Diabetes Metab Res Rev 25, 325-328 (2009).
18. C. J. Greenbaum, A. M. Anderson, L. M. Dolan, E. J. Mayer-Davis, D. Dabelea, G. Imperatore, S. Marcovina, C. Pihoker, S. S. Group, Preservation of beta-cell function in autoantibody-positive youth with diabetes. Diabetes Care 32, 1839-1844 (2009).
19. W. Hagopian, R. J. Ferry, Jr., N. Sherry, D. Carlin, E. Bonvini, S. Johnson, K. E. Stein, S. Koenig, A. G. Daifotis, K. C. Herold, J. Ludvigsson, I. Protege Trial, Teplizumab preserves C-peptide in recent-onset type 1 diabetes: two-year results from the randomized, placebo-controlled Protege trial. Diabetes 62, 3901-3908 (2013).
20. K. C. Herold, S. E. Gitelman, M. R. Ehlers, P. A. Gottlieb, C. J. Greenbaum, W. Hagopian, K. D. Boyle, L. Keyes-Elstein, S. Aggarwal, D. Phippard, P. H. Sayre, J. McNamara, J. A. Bluestone, A. T. E. S. T. Ab, Teplizumab (anti-CD3 mAb) treatment preserves C-peptide responses in patients with new-onset type 1 diabetes in a randomized controlled trial: metabolic and immunologic features at baseline identify a subgroup of responders. Diabetes 62, 3766-3774 (2013).
21. K. C. Herold, W. Hagopian, J. A. Auger, E. Poumian-Ruiz, L. Taylor, D. Donaldson, S. E. Gitelman, D. M. Harlan, D. Xu, R. A. Zivin, J. A. Bluestone, Anti-CD3 monoclonal antibody in new-onset type 1 diabetes mellitus. N Engl J Med 346, 1692-1698. (2002).
22. B. Keymeulen, E. Vandemeulebroucke, A. G. Ziegler, C. Mathieu, L. Kaufman, G. Hale, F. Gorus, M. Goldman, M. Walter, S. Candon, L. Schandene, L. Crenier, C. De Block, J. M. Seigneurin, P. De Pauw, D. Pierard, I. Weets, P. Rebello, P. Bird, E. Berrie, M. Frewin, H. Waldmann, J. F. Bach, D. Pipeleers, L. Chatenoud, Insulin needs after CD3-antibody therapy in new-onset type 1 diabetes. N Engl J Med 352, 2598-2608 (2005).
23. S. A. Long, J. Thorpe, H. A. DeBerg, V. Gersuk, J. Eddy, K. M. Harris, M. Ehlers, K. C. Herold, G. T. Nepom, P. S. Linsley, Partial exhaustion of CD8 T cells and clinical response to teplizumab in new-onset type 1 diabetes. Sci Immunol 1, (2016).
24. N. Sherry, W. Hagopian, J. Ludvigsson, S. M. Jain, J. Wahlen, R. J. Ferry, Jr., B. Bode, S. Aronoff, C. Holland, D. Carlin, K. L. King, R. L. Wilder, S. Pillemer, E. Bonvini, S. Johnson, K. E. Stein, S. Koenig, K. C. Herold, A. G. Daifotis, I. Protege Trial, Teplizumab for treatment of type 1 diabetes (Protege study): 1-year results from a randomised, placebo-controlled trial. Lancet 378, 487-497 (2011).
25. J. E. Tooley, N. Vudattu, J. Choi, C. Cotsapas, L. Devine, K. Raddassi, M. R. Ehlers, J. G. McNamara, K. M. Harris, S. Kanaparthi, D. Phippard, K. C. Herold, Changes in T-cell subsets identify responders to FcR-nonbinding anti-CD3 mAb (teplizumab) in patients with type 1 diabetes. Eur J Immunol 46, 230-241 (2016).
26. K. C. Herold, B. N. Bundy, S. A. Long, J. A. Bluestone, L. A. DiMeglio, M. J. Dufort, S. E. Gitelman, P. A. Gottlieb, J. P. Krischer, P. S. Linsley, J. B. Marks, W. Moore, A. Moran, H. Rodriguez, W. E. Russell, D. Schatz, J. S. Skyler, E. Tsalikian, D. K. Wherrett, A. G. Ziegler, C. J. Greenbaum, G. Type 1 Diabetes TrialNet Study, An Anti-CD3 Antibody, Teplizumab, in Relatives at Risk for Type 1 Diabetes. N Engl J Med 381, 603-613 (2019).
27. K. Näntö-Salonen, A. Kupila, S. Simell, H. Siljander, T. Salonsaari, A. Hekkala, S. Korhonen, R. Erkkola, J. I. Sipilä, L. Haavisto, Nasal insulin to prevent type 1 diabetes in children with HLA genotypes and autoantibodies conferring increased risk of disease: a double-blind, randomised controlled trial. The Lancet 372, 1746-1755 (2008).
28. G. Diabetes Prevention Trial—Type 1 Diabetes Study, Effects of insulin in relatives of patients with type 1 diabetes mellitus. N Engl J Med 346, 1685-1691 (2002).
29. G. Writing Committee for the Type 1 Diabetes TrialNet Oral Insulin Study, J. P. Krischer, D. A. Schatz, B. Bundy, J. S. Skyler, C. J. Greenbaum, Effect of Oral Insulin on Prevention of Diabetes in Relatives of Patients With Type 1 Diabetes: A Randomized Clinical Trial. JAMA 318, 1891-1902 (2017).
30. H. Elding Larsson, M. Lundgren, B. Jonsdottir, D. Cuthbertson, J. Krischer, A.-I. T. S. G. Di, Safety and efficacy of autoantigen-specific therapy with 2 doses of alum-formulated glutamate decarboxylase in children with multiple islet autoantibodies and risk for type 1 diabetes: A randomized clinical trial. Pediatr Diabetes 19, 410-419 (2018).
31. E. A. Gale, P. J. Bingley, C. L. Emmett, T. Collier, G. European Nicotinamide Diabetes Intervention Trial, European Nicotinamide Diabetes Intervention Trial (ENDIT): a randomised controlled trial of intervention before the onset of type 1 diabetes. Lancet 363, 925-931 (2004).
32. D. L. Eizirik, M. L. Colli, F. Ortis, The role of inflammation in insulitis and beta-cell loss in type 1 diabetes. Nat Rev Endocrinol 5, 219-226 (2009).
33. E. B. Tsai, N. A. Sherry, J. P. Palmer, K. C. Herold, The rise and fall of insulin secretion in type 1 diabetes mellitus. Diabetologia 49, 261-270 (2006).
34. E. Ferrannini, A. Mari, V. Nofrate, J. M. Sosenko, J. S. Skyler, D. P. T. S. Group, Progression to diabetes in relatives of type 1 diabetic patients: mechanisms and mode of onset. Diabetes 59, 679-685 (2010).
35. K. C. Herold, S. Usmani-Brown, T. Ghazi, J. Lebastchi, C. A. Beam, M. D. Bellin, M. Ledizet, J. M. Sosenko, J. P. Krischer, J. P. Palmer, G. Type 1 Diabetes TrialNet Study, beta cell death and dysfunction during type 1 diabetes development in at-risk individuals. J Clin Invest 125, 1163-1173 (2015).
36. N. A. Sherry, E. B. Tsai, K. C. Herold, Natural history of beta-cell function in type 1 diabetes. Diabetes 54 Suppl 2, S32-39 (2005).
37. K. C. Herold, W. Hagopian, J. A. Auger, E. Poumian-Ruiz, L. Taylor, D. Donaldson, S. E. Gitelman, D. M. Harlan, D. Xu, R. A. Zivin, J. A. Bluestone, Anti-CD3 monoclonal antibody in new-onset type 1 diabetes mellitus. N Engl J Med 346, 1692-1698 (2002).
38. K. C. Herold, S. E. Gitelman, U. Masharani, W. Hagopian, B. Bisikirska, D. Donaldson, K. Rother, B. Diamond, D. M. Harlan, J. A. Bluestone, A Single Course of Anti-CD3 Monoclonal Antibody hOKT3{gamma}1(Ala-Ala) Results in Improvement in C-Peptide Responses and Clinical Parameters for at Least 2 Years after Onset of Type 1 Diabetes. Diabetes 54, 1763-1769 (2005).
39. L. M. McLane, M. S. Abdel-Hakeem, E. J. Wherry, CD8 T Cell Exhaustion During Chronic Viral Infection and Cancer. Annu Rev Immunol 37, 457-495 (2019).
40. J. M. Sosenko, J. P. Palmer, C. J. Greenbaum, J. Mahon, C. Cowic, J. P. Krischer, H. P. Chase, N. H. White, B. Buckingham, K. C. Herold, D. Cuthbertson, J. S. Skyler, Patterns of metabolic progression to type 1 diabetes in the Diabetes Prevention Trial-Type 1. Diabetes Care 29, 643-649 (2006).
41. M. M. Bogun, B. N. Bundy, R. S. Goland, C. J. Greenbaum, C-Peptide Levels in Subjects Followed Longitudinally Before and After Type 1 Diabetes Diagnosis in TrialNet. Diabetes Care 43, 1-8 (2020).
42. L. Chatenoud, J. Primo, J. F. Bach, CD3 antibody-induced dominant self tolerance in overtly diabetic NOD mice. J Immunol 158, 2947-2954 (1997).
43. M. B. Davidson, A. L. Peters, D. L. Schriger, An alternative approach to the diagnosis of diabetes with a review of the literature. Diabetes Care 18, 1065-1071 (1995).
44. E. M. Akirav, M. T. Baquero, L. W. Opare-Addo, M. Akirav, E. Galvan, J. A. Kushner, D. L. Rimm, K. C. Herold, Glucose and inflammation control islet vascular density and beta-cell function in NOD mice: control of islet vasculature and vascular endothelial growth factor by glucose. Diabetes 60, 876-883 (2011).
45. N. A. Sherry, J. A. Kushner, M. Glandt, T. Kitamura, A. M. Brillantes, K. C. Herold, Effects of autoimmunity and immune therapy on beta-cell turnover in type 1 diabetes. Diabetes 55, 3238-3245 (2006).
46. A. L. Perdigoto, P. Preston-Hurlburt, P. Clark, S. A. Long, P. S. Linsley, K. M. Harris, S. E. Gitelman, C. J. Greenbaum, P. A. Gottlieb, W. Hagopian, A. Woodwyk, J. Dziura, K. C. Herold, N. Immune Tolerance, Treatment of type 1 diabetes with teplizumab: clinical and immunological follow-up after 7 years from diagnosis. Diabetologia 62, 655-664 (2019).
47. K. C. Herold, S. L. Bucktrout, X. Wang, B. W. Bode, S. E. Gitelman, P. A. Gottlieb, J. Hughes, T. Joh, J. B. McGill, J. H. Pettus, S. Potluri, D. Schatz, M. Shannon, C. Udata, G. Wong, M. Levisetti, B. J. Ganguly, P. D. Garzone, R. N. W. Group, Immunomodulatory activity of humanized anti-IL-7R monoclonal antibody RN168 in subjects with type 1 diabetes. JCI Insight 4, (2019).
48. M. Battaglia, M. S. Anderson, J. H. Buckner, S. M. Geyer, P. A. Gottlieb, T. W. H. Kay, A. Lernmark, S. Muller, A. Pugliese, B. O. Roep, C. J. Greenbaum, M. Peakman, Understanding and preventing type 1 diabetes through the unique working model of TrialNet. Diabetologia 60, 2139-2147 (2017).
49. L. Yu, D. C. Boulware, C. A. Beam, J. C. Hutton, J. M. Wenzlau, C. J. Greenbaum, P. J. Bingley, J. P. Krischer, J. M. Sosenko, J. S. Skyler, G. S. Eisenbarth, J. L. Mahon, G. Type 1 Diabetes TrialNet Study, Zinc transporter-8 autoantibodies improve prediction of type 1 diabetes in relatives positive for the standard biochemical autoantibodies. Diabetes Care 35, 1213-1218 (2012).
50. A. American Diabetes, 2. Classification and Diagnosis of Diabetes: Standards of Medical Care in Diabetes 2019. Diabetes Care 42, S13-S28 (2019).
51. C. Steele, W. A. Hagopian, S. Gitelman, U. Masharani, M. Cavaghan, K. I. Rother, D. Donaldson, D. M. Harlan, J. Bluestone, K. C. Herold, Insulin Secretion in Type 1 Diabetes. Diabetes 53, 426-433 (2004).
52. K. S. Polonsky, J. Licinio-Paixao, B. D. Given, W. Pugh, P. Rue, J. Galloway, T. Karrison, B. Frank, Use of biosynthetic human C-peptide in the measurement of insulin secretion rates in normal volunteers and type I diabetic patients. J Clin Invest 77, 98-105 (1986).
53. E. Van Cauter, F. Mestrez, J. Sturis, K. S. Polonsky, Estimation of insulin secretion rates from C-peptide levels. Comparison of individual and standard kinetic parameters for C-peptide clearance. Diabetes 41, 368-377 (1992).
54. S. A. Long, J. Thorpe, K. C. Herold, M. Ehlers, S. Sanda, N. Lim, P. S. Linsley, G. T. Nepom, K. M. Harris, Remodeling T cell compartments during anti-CD3 immunotherapy of type 1 diabetes. Cell Immunol 319, 3-9 (2017).

1. A method of preventing or delaying the onset of clinical type 1 diabetes (T1D), comprising:
providing a non-diabetic subject who is at risk for T1D;
administering a prophylactically effective amount of an anti-CD3 antibody to the non-diabetic subject; and
determining, prior to or after the administering step, that the non-diabetic subject has more than about 5% to more than about 10% TIGIT+KLRG1+CD8+ T-cells in all CD3+ T-cells, which is indicative of successful prevention or delay of the onset of clinical T1D.

2-13. (canceled)

14. A method of measuring responsiveness to an anti-CD3 antibody in preventing or delaying the onset of clinical type 1 diabetes (T1D), comprising:
administering a prophylactically effective amount of an anti-CD3 antibody to a non-diabetic subject who is at risk for T1D;
determining C-peptide area under the curve (AUC): glucose AUC ratio, wherein an increase in said ratio indicates responsiveness to the anti-CD3 antibody in preventing or delaying progression to clinical T1D, and optionally repeating administration of the anti-CD3 antibody at 2 month, 4 month, 6 month, 8 month, 9 month, 10 month, 12 month, 15 month, 18 month, 24 month, 30 month, or 36 month intervals if responsiveness to the anti-CD3 antibody is found.

15. The method of claim 14, wherein the non-diabetic subject is a relative of a patient with T1D.

16. The method of claim 14, wherein the non-diabetic subject (1) is substantially free of antibodies against zinc transporter 8(ZnT8), (2) is HLA-DR4+, and/or (3) is not HLA-DR3+.

17. The method of claim 16, wherein the non-diabetic subject has 2 or more diabetes-related autoantibodies selected from islet cell antibodies (ICA), insulin autoantibodies (IAA), glutamic acid decarboxylase (GAD) antibodies, tyrosine phosphatase (IA-2/ICA512) antibodies, and ZnT8 antibodies.

18. The method of claim 14, wherein the non-diabetic subject has abnormal glucose tolerance on oral glucose tolerance test (OGTT).

19. The method of claim 16, wherein the non-diabetic subject does not have antibodies against ZnT8.

20. The method of claim 16, wherein the non-diabetic subject is HLA-DR4+ and is not HLA-DR3+.

21. The method of claim 14, wherein the anti-CD3 antibody is teplizumab, otelixizumab, or foralumab.

22. The method of claim 21, wherein the prophylactically effective amount comprises a 10 to 14 day course of subcutaneous (SC) injection or intravenous (IV) infusion of the anti-CD3 antibody at 10-1000 micrograms/meter squared (μg/m²), preferably a 14-day course of IV infusion at 51 μg/m², 103 μg/m², 207 μg/m², and 413 μg/m², on days 0-3, respectively, and one dose of 826 μg/m² on each of days 4-13.

23. The method of claim 21, wherein the prophylactically effective amount comprises a 10 to 14 day course of subcutaneous (SC) injection or intravenous (IV) infusion of the anti-CD3 antibody at a total dose of from about 9034 μg/m² to about 14634 μg/m².

24. The method of claim 14, wherein the determining step is carried out at three months after the administering step.

25. A method of increasing the proportion of regulatory T-cells and of exhausted CD8+ T-cells in peripheral blood of a Stage 2 type 1 diabetes (T1D) patient, comprising administering a prophylactically effective amount of an anti-CD3 antibody to the patient.

26. The method of claim 25, wherein the Stage 2 T1D patient is a relative of a patient with clinical T1D.

27. The method of claim 25, wherein the Stage 2 T1D patient has two or more autoantibodies selected from islet cell autoantibodies (ICA), insulin autoantibodies (IAA), glutamic acid decarboxylase (GAD) autoantibodies, tyrosine phosphatase (IA-2/ICA512) autoantibodies, and ZnT8 autoantibodies.

28. The method of claim 25, wherein the Stage 2 T1D patient has abnormal glucose tolerance on oral glucose tolerance test (OGTT).

29. The method of claim 25, wherein the anti-CD3 antibody is teplizumab, otelixizumab, or foralumab.

30. The method of claim 25, wherein the prophylactically effective amount comprises a 10 to 14 day course of subcutaneous (SC) injection or intravenous (IV) infusion of the anti-CD3 antibody at a total dose of from about 9034 μg/m² to about 14634 μg/m².

31. The method of claim 25, wherein the prophylactically effective amount comprises a 10 to 14 day course of subcutaneous (SC) injection, intravenous (IV) infusion, of the anti-CD3 antibody at 10-1000 micrograms/meter squared (μg/m²), preferably a 14-day course of IV infusion at 51 μg/m², 103 μg/m², 207 μg/m², and 413 μg/m², on days 0-3, respectively, and one dose of 826 μg/m² on each of days 4-13.

32. The method of claim 25, wherein the exhausted CD8+ T-cells express TIGIT and KLRG1.

33. The method of claim 25, wherein the expression of Ki67 and CD57 in the CD8+ T-cells is lowered.
